(19) United States
(12) Patent Application Publication
(10) Pub. No.: US 2006/0030876 A1
(43) Pub. Date: Feb. 9, 2006

(54) Embolic filter device and related systems and methods

(76) Inventors: James C. Peacock III, San Carlos, CA (US); Lawrence Wasicek, San Jose, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/119,398

(22) Filed: Apr. 29, 2005

Related U.S. Application Data
(63) Continuation of application No. PCT/US03/34266, filed on Oct. 28, 2003.
(60) Provisional application No. 60/421,975, filed on Oct. 29, 2002.

(51) Int. Cl. A61M 29/00

(52) U.S. Cl. 606/200

(57) ABSTRACT

An embolic filter system includes a filter device that is adapted to be positioned over a guidewire and locked onto the guidewire at the position. The filter device may be locked over the guidewire outside of the patient, and then the locked assembly is advanced within a delivery sheath to the desired filtering location where the filter is adjusted to an expanded configuration to filter emboli there. Or, the filter device in a radially collapsed configuration may be advanced over an indwelling guidewire by back-loading the guidewire into a guidewire lumen associated with the filter device. Once delivered to the desired filtering location along the guidewire, the filter is locked onto the guidewire and then expanded to the radially expanded configuration for filtering the emboli. A control system cooperates with the embolic filter device to selectively lock the embolic filter device onto the guidewire and to selectively adjust the filter assembly between radially collapsed and expanded configurations for delivery and filtering, respectively. The control system includes a locking system, which in one beneficial embodiment has at least one shape memory member that is adapted to be heated and reshaped to thereby lock onto the guidewire.

### CROSS-REFERENCE TO RELATED APPLICATIONS

This application claims priority from, and is a 35 U.S.C. § 111(a) continuation of, co-pending PCT international application serial number PCT/US2003/034266 filed on Oct. 28, 2003, which designates the U.S., incorporated herein by reference in its entirety, which in turn claims priority from U.S. provisional application Ser. No. 60/421,975 filed on Oct. 29, 2002, incorporated herein by reference in its entirety. Priority is claimed to each of the foregoing applications.

The foregoing referenced PCT international application was published as International Publication No. WO 2004/039287 A2 on May 13, 2004, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

The present invention is a system and method for filtering emboli from fluid flowing through a body lumen in a patient. More specifically, it is an embolic filter system and method adapted for adjustable use over an indwelling guidewire for filtering emboli from blood flowing through a blood vessel in a patient. This invention pertains generally to

2. Description of Related Art

Several embolic filter technologies have been disclosed for filtering emboli released during interventional procedures. One particular circumstance where embolic filtering has been investigated is for distal protection against emboli flowing toward the brain during carotid artery interventions, such as endarterectomy, angioplasty, stenting, or atherectomy or rotational ablation. Another circumstance under investigation is filtering distal run off of emboli during recanalization of grafts, such as coronary bypass grafts.

In general, distal embolic protection systems and methods provide a filter pre-disposed on a distal end portion of a guidewire chassis. The guidewire and filter are positioned translumenally through and across the intervention site in an antegrade fashion so that the filter is positioned downstream from the occlusion to be recanalized. Then the filter is deployed, generally as an expanded cage or porous material that allows blood to pass but for emboli of a predetermined size (according to the passage ports, e.g. through pores or other openings in the filter). The intervention upstream from the filter releases emboli that flow downstream into the deployed filter where they are caught. After the intervention is complete, a mechanism is provided that allows the filter to be adjusted for withdrawal, including capturing the emboli caught.

Further examples of devices and methods that provide additional background helpful in understanding the overall context of the present invention are provided in the following U.S. patents: U.S. Pat. No. 6,027,520 to Tsugita et al.; U.S. Pat. No. 6,042,598 to Tsugita et al.; U.S. Pat. No. 6,168,579 to Tsugita; U.S. Pat. No. 6,270,513 to Tsugita et al.; U.S. Pat. No. 6,277,139 to Levinson et al.; and U.S. Pat. No. 6,319,242 to Patterson et al. Additional examples are disclosed in the following International Publications: WO 00/67664 to Salviac Limited; WO 01/49215 to Advanced Cardiovascular Systems, Inc.; WO 01/80777 to Salviac Limited; and WO 02/43595 to Advanced Cardiovascular Systems, Inc. The disclosures of the documents in this paragraph are herein incorporated in their entirety by reference thereto.

### BRIEF SUMMARY OF THE INVENTION

The present invention is an embolic filter system that includes an embolic filter device that is adapted to be used over a guidewire such that the guidewire is provided independent of, though cooperates with, the filter device.

In one aspect, the embolic filter device is adjustable between a first configuration and a second configuration, and also between unlocked and locked conditions with respect to the guidewire. In the first configuration and unlocked condition, the embolic filter device is adapted to be slideably positioned over the guidewire at a position where filtering is desired. The filter device is adapted to be adjusted to the locked condition onto the wire at the position. The filter device is further adapted to be adjusted in-vivo to the second configuration that is adapted to filter emboli from fluids flowing therethrough at a filtering location corresponding to the filter device's locked position along the guidewire.

In one mode, the filter device is adapted to filter emboli from blood. In one embodiment, the device is adapted to be positioned with the guidewire downstream from an intervention site in a carotid artery in a patient and to filter emboli released during the intervention at the intervention site.

In another embodiment, the filter system is adapted to be positioned downstream from an anastomosed arterial or venous graft, and is adapted to filter emboli from blood flowing downstream from the graft, such as during an intervention such as recanalization of the graft.

In another mode, the filter device has a filter assembly secured onto a tubular support member. The tubular support member has a guidewire passageway therethrough and is adjustable between a first configuration and a second configuration. In the first configuration the guidewire passageway has a first inner diameter that is adapted to allow the tubular support member to be moveably engaged over the guidewire for adjustable placement of the filter device along the length of the guidewire. In the second configuration, the guidewire passageway has a second inner diameter that is adapted to engage the guidewire sufficient to lock the filter device onto the guidewire such that the filter device remains on the guidewire during in-vivo use.

In another mode, the filter device adjusts to the second configuration in response to an applied energy. In one embodiment, the filter device is adapted to adjust to the second configuration in response to an applied electrical current to a conductor associated with the filter device. In another embodiment, the filter device is adapted to adjust to the second configuration in response to applied ultrasound energy. In another embodiment, the adjustment is in response to an applied light energy.

In another mode, the filter system includes a control system coupled to the filter device and that is adapted to control the positioning, locking, and radial adjusting of the filter device with respect to a guidewire.

According to one embodiment of this mode, the control system includes a delivery member that is adapted to hold the filter device and advance the filter device over a guidewire to the position where it is desired to be locked. The control system in another embodiment includes a lock member that is adapted to lock the filter device at the position along the guidewire.

In another embodiment, the control system includes a radial adjusting system that is adapted to couple to the filter device and adjust it between the first and second configurations. In one variation of this embodiment, the radial adjusting system includes an outer sheath that is longitudinally moveable over the guidewire between first and second positions, respectively, with respect to the filter device. In the first position, the filter device is radially contained within a passageway of the outer sheath in a radially collapsed condition. In the second position, the filter device is located exteriorly of the passageway and is adapted to expand to a memory state that is a radially expanded condition corresponding to the second configuration. In another variation, a pull wire is coupled to a radial support member.

In another aspect, the invention is an embolic filter system with a filter device that includes a filter assembly with a radial support member coupled to a filter wall. In a radially expanded condition, the radial support member supports at least in part the filter wall in a shape that is adapted to filter blood flowing into the assembly of the radially support member and wall.

In one mode, the filter wall is a sheet of material. In one embodiment, the sheet of material comprises a porous membrane with pores having sufficient size to allow normal physiological blood components to pass therethrough, but to filter larger components such as emboli from passing. In another embodiment, the sheet of material has a plurality of apertures formed therethrough.

In another mode, the filter wall is a meshed network of strand material having spaces between strands of sufficient size to allow normal physiological blood components to pass therethrough, but to filter larger components such as emboli from passing.

The invention in another aspect is an embolic filter system having an embolic filter device coupled to a control system that includes at least one detachable member that is detachable from the embolic filter device when the embolic filter device is positioned at a remote in-vivo location.

In one mode of this aspect, the detachable member is a conductor lead that is adapted to couple energy from an ex-vivo energy source to the embolic filter device at the remote in-vivo location. In one embodiment of this mode, the conductor lead is electrolytically detachable from the filter device upon application of sufficient electrical energy to a sacrificial link between the conductor lead and the filter device.

The invention in another aspect is an embolic filter system with an embolic filter device that includes a filter assembly coupled to a locking member. The locking member is adjustable between an unlocked condition and a locked condition. In the unlocked condition, the filter device is adapted to be advanced over a guidewire to a desired position. In the locked condition, the filter device is substantially locked onto the guidewire at the position.

The invention in another aspect is an embolic filter system with an embolic filter device that includes a filter assembly cooperating with an adjustable member. The adjustable member is adjustable between a first shape and a second shape. In the first shape the adjustable member is allow for passage of a guidewire therethrough. In the second shape, the filter device is adapted to be locked onto the guidewire.

In one mode, the adjustable member has a first inner diameter in the first shape, and a second inner diameter that is smaller than the first inner diameter in the second shape.

In another mode, the adjustable member is formed at least in part from a shape-memory material. In one embodiment, the shape memory material is nickel-titanium alloy. In one variation, the nickel-titanium alloy forms an annular member such as a ring. In a further feature, the ring may have a memory state in the second shape. In a further feature, the ring is adjustable between the first and second shapes at a particular temperature. In a further feature, the temperature is above normal resting body temperature.

In another mode, the adjustable member is adapted to be positioned along the guidewire and has a first outer diameter in the first shape and a second outer diameter in the second shape. The first outer diameter is sufficiently small to slideable clearance between the guidewire at the position of the adjustable member and a guidewire passageway of the filter device. The second outer diameter is larger than the first outer diameter and is sufficient to radially engage the guidewire passageway to thereby lock the filter device onto the guidewire at the position of the adjustable member.

The invention according to another aspect is an embolic filter system with an embolic filter device having a filter assembly cooperating with an annular member that is adjustable between first and second inner diameters. The first inner diameter is greater than an outer diameter of the guidewire. The second inner diameter is less than the outer diameter of the guidewire.

In one mode, the annular member is formed at least in part from a shape-memory material. In one embodiment, the shape memory material is nickel-titanium alloy.

In another mode, the annular member is a ring.

In another mode, the annular member is a coil.

In another mode, the annular member is a tubular member.

In another mode, the annular member comprises a pattern of interconnected struts separated by void areas.

In another mode, the annular member is formed at least in part from a solid tubular member that has a pattern of voids cut therein.

In another mode, the annular member has a memory condition in the second shape. In one embodiment, the annular member is adjustable between the first and second shapes at a transition temperature. In one variation, the transition temperature is above normal resting body temperature. In another variation, the transition temperature is equal to about normal resting body temperature.

The invention according to another aspect is a method for providing an embolic filter system, comprising providing an embolic filter device; placing a distal end portion of a guidewire at a remote in-vivo location within a body of a patient; advancing the filter device over the guidewire in a first configuration and unlocked condition to a position along the distal end portion of the guidewire where filtering is desired; locking the filter device onto the guidewire by adjusting the filter device from the unlocked condition to the locked condition at the position; and adjusting the locked filter device at the position from the first configuration to the second configuration that is adapted to filter emboli from fluid flowing into the filter.

According to one mode of this aspect, the method further includes heating the filter device at the position by coupling the filter device to an energy source located externally from the body; and wherein the heat adjusts the filter device from the unlocked condition to the locked condition. In a further embodiment, the heating includes applying an electrical current to a conductor associated with the filter device, and in one variation the method includes applying an RF current to the conductor. In another embodiment, the heating includes optically coupling light to a conductor associated with the filter that is adapted to heat upon absorbing the light. In another embodiment, the heating includes coupling ultrasound energy to a conductor associated with the filter device that is adapted to heat upon ultrasound absorbance. The ultrasound energy may be produced within the system itself within the body, such as by coupling an ultrasound crystal associated with the filter device with an electrical source externally of the body that is adapted to energize the ultrasound crystal to produce the ultrasound energy.

Another mode of this aspect includes adjusting an adjustable member of the filter device from a first shape to a second shape that correspond with the unlocked and locked conditions, respectively, for the device. In the first shape, there is clearance for the filter device to slideably engage and move over the guidewire. In the second shape, the adjustable member engages the guidewire. In one embodiment the adjusting includes reducing the inner diameter of an annular ring. In another embodiment, the adjusting includes reducing the inner diameter of a longitudinally extending coil or braid.

The invention in another aspect provides an embolic filter as a module that is adapted to be removably engaged onto a guidewire.

The invention in another aspect provides an embolic filter that is adapted to be delivered over an indwelling guidewire, positioned at a location along a distal end portion of the guidewire distal to a site of intervention, and locked onto the guidewire at the location.

The invention according to another aspect provides an embolic filter that is adjustable between radially collapsed and radially expanded conditions on a guidewire positioned at a location distal to an intended invention site.

The invention also includes various aspects that are adaptations of the aspects, modes, embodiments, variations, and features above as a proximal embolic filtering system and method.

Another aspect of the invention is an embolic filter system with a filter assembly and an adjustable lock assembly as follows. The filter assembly has a filter member that is adjustable between a radially collapsed configuration and a radially expanded configuration. The filter assembly is adapted to be locked with the adjustable lock assembly at a selected position along a distal end portion of a guidewire at a location within a lumen in a patient's body, and is adapted to be delivered at least in part with the guidewire to the location in the locked configuration. The filter member is adjustable at the location from the radially collapsed configuration to a radially expanded configuration that spans across a substantial cross-section of the lumen. The filter member in the radially expanded configuration at the location is also adapted to filter components of fluid flowing through the lumen at the location above a predetermined size.

Another aspect of the invention is an embolic filter system with a delivery member that cooperates with a filter assembly as follows. The delivery member has an elongate body having a proximal end portion and a distal end portion. The filter assembly has a filter member that is adjustable between a radially collapsed configuration and a radially expanded configuration. The distal end portion of the delivery member is coupled to the filter assembly and is adapted to at least in part advance the filter assembly in the radially collapsed configuration to a location within a lumen in a body of a patient by manipulating the proximal end portion externally of the patient's body. The filter member is adjustable at the location from the radially collapsed configuration to a radially expanded configuration that spans across a substantial cross-section of the lumen. The filter member in the radially expanded configuration at the location is adapted to filter components of fluid flowing through the lumen at the location above a predetermined size. The distal end portion of the delivery member is detachable from the filter assembly at the location.

Another aspect of the invention is an embolic filter system with a delivery member, a filter assembly, and an adjustable lock assembly as follows. The delivery member has an elongate body having a proximal end portion and a distal end portion. The filter assembly includes a guidewire tracking member, and a filter member coupled to the guidewire tracking member and that is adjustable between a radially collapsed configuration and a radially expanded configuration. The distal end portion of the delivery member is detachably coupled to the guidewire tracking member and is adapted to advance the filter assembly with the filter member in the radially collapsed configuration over the guidewire to the location by manipulating the proximal end portion of the delivery member externally of the patient's body. The filter member is adjustable at the location from the radially collapsed configuration to a radially expanded configuration that spans across a substantial cross-section of the lumen. The filter member in the radially expanded configuration at the location is adapted to filter components of fluid flowing through the lumen at the location above a predetermined size. The adjustable lock assembly is adapted to lock the filter assembly onto the distal end portion of the guidewire at the location, and the delivery member is detachable from the guidewire tracking member at the location.

Another aspect of the invention is an embolic filter system with a delivery assembly that cooperates with a filter assembly as follows. The filter assembly has a filter member having a wall with a substantially annular passageway around a circumference, and with a superelastic loop-shaped member coupled to the filter member within the annular passageway and along the circumference. The superelastic loop-shaped support member is adjustable between a radially collapsed condition corresponding with an elastically deformed condition for the loop-shaped member and a radially expanded condition according to material recovery from the elastically deformed condition to a memory condition. Adjusting the support member from the radially collapsed condition to the radially expanded condition adjusts the filter member between a radially collapsed configuration and a radially expanded configuration, respectively. The filter assembly is adapted to be delivered at least in part with the delivery assembly to a location within a lumen in a body of a patient with the support member radially confined in the radially collapsed condition and the filter member in the radially collapsed configuration. The support member and filter member are adjustable from the radially collapsed condition and radially collapsed configuration, respectively, to the radially expanded configuration and radially expanded configuration, also respectively, at the location. The filter member in the radially expanded configuration at the location spans across a substantial cross-section of the lumen. The filter member in the radially expanded configuration at the location is adapted to filter components of fluid flowing through the lumen at the location above a predetermined size.

Another aspect of the invention is an embolic filter system as follows. The system includes a delivery member with an elongate body having a proximal end portion and a distal end portion with a longitudinal axis, and a lumen extending between proximal and distal ports each being located along the distal end portion. The system also includes a filter assembly with a filter member coupled to a support member and that is adjustable from a radially collapsed configuration corresponding with an elastically deformed condition for the filter member and to a radially expanded configuration according to memory recovery from the elastically deformed condition toward a memory condition. The filter assembly in the radially collapsed configuration is radially confined within the lumen and is adapted to be delivered to a location within a lumen in a body of a patient. The filter assembly is adjustable from the radially collapsed configuration at the location to the radially expanded configuration at the location by removal of the filter assembly from the radially confining lumen. The filter member in the radially expanded configuration at the location spans across a substantial cross-section of the lumen, and is adapted to filter components of fluid flowing through the lumen at the location above a predetermined size.

Another aspect of the invention is a method for filtering emboli from fluid flowing across a location within a body lumen in a patient that includes the following steps. A filter assembly is delivered in a radially collapsed configuration over a guidewire to the location. The filter assembly is locked onto the guidewire at the location, and is then adjusted from the radially collapsed configuration to a radially expanded configuration at the location. The filter assembly in the radially expanded configuration at the location spans across a substantial cross-section of the body lumen and is adapted to filter the emboli from the fluid flowing across the location.

Another aspect of the invention is a method for filtering emboli from fluid flowing across a location within a body lumen in a patient as follows. A filter assembly is delivered with a delivery member in a radially collapsed configuration over a guidewire to the location. The filter assembly is detached from the delivery member at the location. The filter assembly is adjusted from the radially collapsed configuration to a radially expanded configuration at the location, which spans across a substantial cross-section of the body lumen and is adapted to filter the emboli from the fluid flowing across the location. The filter assembly is thereafter collapsed with filtered emboli captured therewith. Then, the collapsed filter assembly is removed from the body lumen.

Another aspect of the invention is another method for filtering emboli from fluid flowing across a location within a body lumen in a patient as follows. A filter assembly is positioned in a radially collapsed configuration within a capture lumen of a radially confining cuff having an adjustable position relative to the filter assembly. The filter assembly is provided in the radially collapsed configuration within the adjustable radially confining cuff along a distal end portion of a delivery member. The distal end portion of the delivery member and filter assembly are delivered in the radially collapsed condition within the cuff to the location, and the filter assembly is adjusted from the radially collapsed configuration to a radially expanded configuration at the location by adjusting the relative position of the cuff relative to the filter assembly such that the filter assembly is released from radial confinement and self-expands according to material memory to the radially expanded condition. The filter assembly in the radially expanded configuration at the location spans across a substantial cross-section of the body lumen and is adapted to filter the emboli from the fluid flowing across the location. The filter assembly is thereafter collapsed with filtered emboli captured therewith by positioning the filter assembly at least in part back within the radially confining cuff, and is removed at least partially confined within the cuff from the body lumen. Further to this method, the capture lumen extends along a length between proximal and distal ports and is located entirely within the body lumen, such as for example when the filter assembly is located within the cuff to the location.

Another aspect of the invention is a method for assembling an embolic filter system as follows. A guidewire is provided that has a proximal end portion and a distal end portion with a first length that is adapted to be positioned at a location within a lumen in a patient while the proximal end portion extends externally from the patient. A filter assembly is also provided with a filter member coupled to a guidewire tracking member having a guidewire lumen extending with a second length between a proximal port and a distal port. The guidewire lumen is slideably engaged over the guidewire. The second length is less than the first length, such that the filter assembly is a shuttle that tracks over the guidewire. The shuttling filter assembly according to a further mode is locked onto the distal end portion of the guidewire.

The various aspects, modes, embodiments, variations, and features just described are to be considered independently beneficial without requiring limitation by the others. However, further combinations and sub-combinations apparent to one of ordinary skill are also contemplated as within the scope of the present invention. Other beneficial aspects, modes, and embodiments are to be appreciated by one of ordinary skill based upon further review of the disclosure below and accompanying Figures.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

FIGS. 1A-D show plan schematic views of 4 sequential modes, respectively, of using the invention for attaching an embolic filter onto a guidewire before placing the attached assembly into the body of a patient.

FIGS. 2A-B show side views of the distal end portion of one particular assembly of the invention wherein a distal embolic filter module is engaged over a guidewire in radially expanded and collapsed conditions, respectively.

FIGS. 3A-B show side views of the distal end portion of another particular assembly of the invention wherein a distal embolic filter module is engaged over a guidewire in radially expanded and collapsed conditions, respectively.

FIGS. 4A-B longitudinally cross-sectioned side views of another embolic filter assembly of the invention with a tubular support member coaxially engaged over a guidewire in a radially expanded unlocked condition and radially collapsed locked condition, respectively, with respect to the guidewire.

FIGS. 5A-B show longitudinally cross-sectioned side views of another embolic filter embodiment in radially expanded unlocked and radially collapsed locked conditions, respectively, with respect to a coaxially engaged guidewire shown schematically extending therethrough.

FIGS. 6A-B longitudinally cross-sectioned side views of another embolic filter embodiment in radially expanded unlocked and radially collapsed locked conditions, respectively, with respect to a coaxially engaged guidewire shown schematically extending therethrough.

FIG. 7 shows a longitudinally cross-sectioned side view of an embolic filtering assembly with an embolic filter device coaxially engaged over a guidewire with an expandable member on the guidewire adjustable between radially collapsed and expanded (shown in shadow) conditions corresponding to locked and unlocked configurations, respectively, between the filter and guidewire.

FIG. 8 shows a longitudinally cross-sectioned side view of a portion of a guidewire chassis that is adapted for use with an assembly of the invention such as that shown in FIG. 7.

FIG. 9A-C show longitudinally cross-sectioned side views of another embolic filter embodiment of the invention in various respective modes of use during a locking procedure over a guidewire.

FIG. 10A shows a partial perspective view of a distal end portion of another embolic filter system of the invention in one mode of use for delivery to a filtering location in a body of a patient.

FIG. 10B shows an exploded perspective view of a distal end portion of a similar filter to that shown in FIG. 10A, and shows in shadow the adjustability of the filter between radially collapsed and expanded conditions, respectively.

FIG. 10C shows an exploded perspective view of a distal end portion of a filter system similar to that shown in FIGS. 10A-B, and shows the outer sheath adjusted to a proximal position such that the filter is adjusted to the radially expanded condition that is adapted to filter emboli from blood in-vivo.

FIG. 10D shows an exploded perspective view of the same distal end portion of the filter system shown in FIG. 10C, except during another mode of use with the filter adjusted back to a radially collapsed condition that captures emboli for removal from the patient's body.

FIGS. 11A-C show partially longitudinally cross-sectioned side view of another embolic filter system of the invention that is adapted to provide in-vivo placement and locking engagement of a filter over an “indwelling” guidewire during various respective modes of use including slideable placement over the guidewire shown in FIG. 11A, locking engagement and detachment shown in FIG. 11B, and radial adjustment to a radially expanded condition for filtering blood shown in FIG. 11C.

FIG. 12 shows a longitudinally cross-sectioned side view of another modified embodiment of the filter system shown in FIGS. 11A-C.

### DETAILED DESCRIPTION OF THE EMBODIMENTS OF THE INVENTION

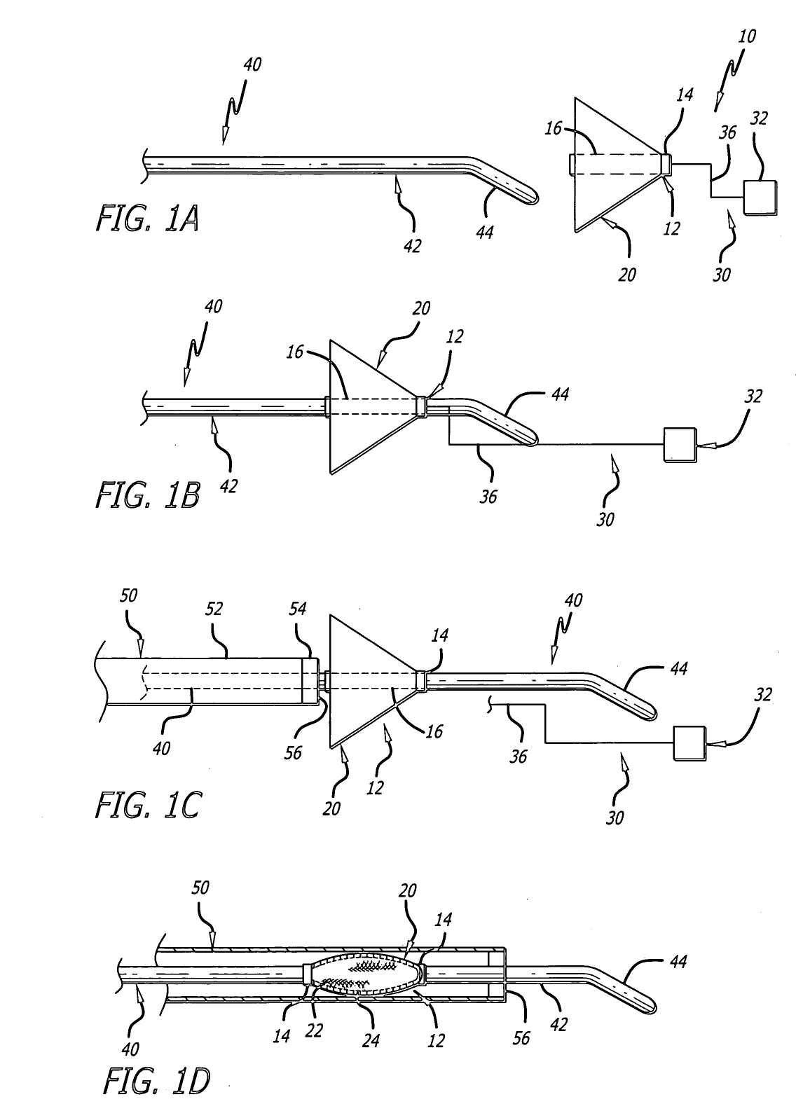

The FIGS. 1-12 variously provide certain details of various beneficial embodiments illustrative of one or more aspects and modes of the invention. While each is considered independently beneficial, additional combinations and sub-combinations between the Figures are also contemplated.

It is to be appreciated according to various of the foregoing embodiments that an embolic filter system is provided that includes an over-the-wire filter assembly coupled to a delivery assembly. The filter assembly has a guidewire tracking assembly that is adapted to slideably engage a guidewire initially placed across a vascular occlusion and is advanced by the delivery assembly in the radially collapsed condition to slide or “shuttle” over the distally seated guidewire and follow the guidewire to the distal filtering location past the vascular occlusion. The filter assembly includes an adjustable lock assembly that is adjustable between an open position, which allows the filter assembly to shuttle over the guidewire, to a locked position, which locks the filter assembly onto the guidewire in situ at the distal location past a vascular occlusion. Once locked onto the guidewire, the filter is adjustable to the radially expanded condition and is detachable from the delivery assembly and thus becomes a part of the guidewire in-situ at the distal location. Thereafter the filter assembly is adapted to be withdrawn in unison with the guidewire and to be groomed into a captured configuration within a capture sheath.

According to other aspects illustrated by various of the embodiments below, a loop-shaped support member is housed within a circumferential passageway formed within a filter member wall. The support member is self-adjustable from a radially collapsed condition to a radially expanded condition that generally correspond with radially collapsed and expanded configurations for the filter member wall. The support member is a memory alloy metal and self-adjusts to the radially expanded condition according to material recovery from a deformed condition of the material corresponding with the radially collapsed condition to a memory condition. The support member is adjusted to the radially collapsed condition within a radial constraint, such as within a delivery lumen of a delivery or guide sheath.

Accordingly, further more detailed embodiments are provided as follows and provide further illustration of the various aspects provided above, as well as other beneficial aspects as is made apparent to one of ordinary skill with this disclosure.

FIGS. 1A-D show various modes of operation according to one embodiment of the invention that provides an embolic filter assembly 10 as follows.

FIG. 1A shows a filter module 12 that is coupled to an actuator assembly 30 and is provided separate from a guidewire 40. Filter module 12 includes a tubular support spine 14 with an inner lumen 16, onto which is coupled an adjustable filter member 20. Actuator assembly 30 includes an actuator 32 and a coupling member 36 that couples actuator 32 to filter module 12.

FIG. 1B shows filter module 12 slideably engaged over distal end portion 42 of guidewire 40 via inner lumen 16 in a “backloading” technique initiated at guidewire tip 44, typically provided as a pre-shaped or shapeable, steering tip. At the desired position along guidewire distal end portion 42, the filter module 12 is actuated via actuator 32 and coupling member 36 to lock onto the guidewire. Once filter module 12 is locked onto guidewire 40, coupling member 36 is detached from filter module 12 and thus filter module 12 and guidewire 40 become an integrated assembly, as shown in FIG. 1C. As further shown in FIG. 1C, this is performed while guidewire 40 is slideably engaged within delivery lumen 56 of delivery sheath 50, and while guidewire distal end portion 42 extends distally from distal tip 54 of the distal end portion 52 of that delivery sheath. However, the assembly of filter module 12 and guidewire 40 may be performed in other manners of operation, such as prior to engaging the guidewire 40 within delivery lumen 56.

As shown in FIG. 1D, once the filter module 12 and guidewire 40 are locked together and coupled, the filter module 12 is adjusted relative to the longitudinal axis L of delivery sheath 50 so as to be positioned within delivery lumen 56, thus collapsing adjustable filter member 20 from a radially expanded condition shown in FIG. 1A-1C to a radially confined condition shown in FIG. 1D. FIG. 1D shows certain further detail of one embodiment for filter member 20 for further illustration, and shows a collapsed configuration for a proximal support member 24 and folded filter wall 22. Proximal support member is for example a ring-shaped support member that is constructed of a superelastic alloy material, such as a nickel-titanium material, having a memory shape corresponding the a radially expanded configuration that further corresponds to the expanded condition of the filter member 20 shown in FIGS. 1A-C. Filter wall 22 is for example a porous sheet of material, or other filter membrane or structure. Further aspects of these respective components will be explained in further detail by reference to other exemplary embodiments below.

It is to be appreciated therefore that the embodiment illustrated by FIGS. 1A-D provide a beneficial ability to customize the position of a filter assembly along a guidewire, such as at a location along its length relative to other structures such as the distal guidewire tip 44. This allows the ability to customize the filtering location in reference to a desired placement of the guidewire tip 44 in the body. Moreover, the filter may be used with a variety of different guidewires, such as stiffer, more flexible, varied tip shapes, varied diameter sizes, materials, etc. The physician is not required to use a particular guidewire provided with the filter. Thus, particular anatomical or procedural concerns specific to a patient intervention may be met with the ability to customize the filtering device. Still further, this arrangement nevertheless allows the guidewire and filter assembly to be integrated ex-vivo prior to the intervention, providing certain other benefits including for example the potential to achieve lower profiles than certain other “over-the-wire” filtering assemblies and techniques that track over a guidewire in-vivo.

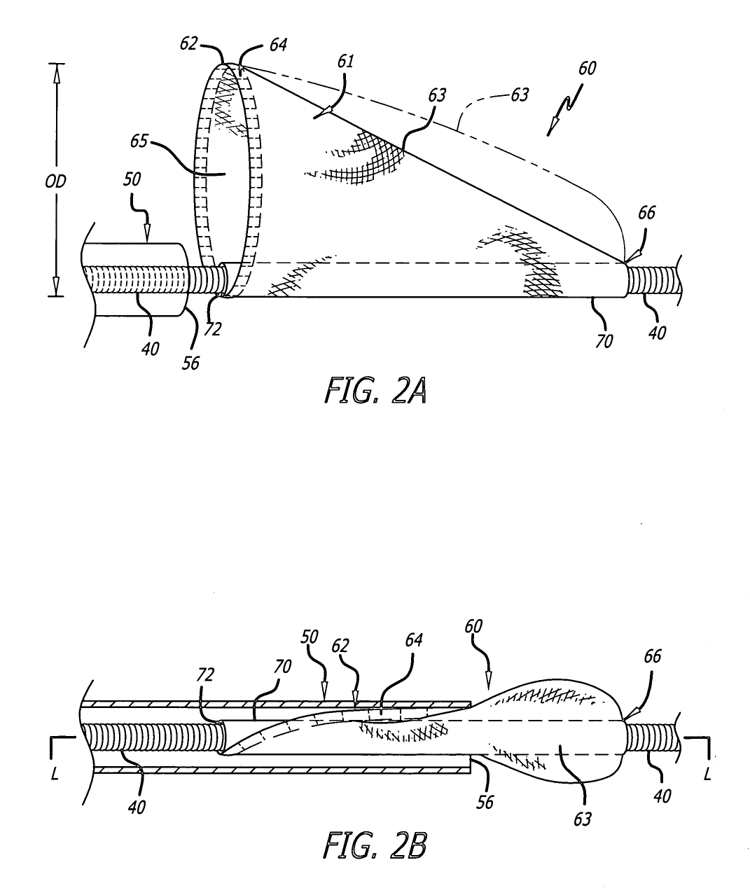

FIGS. 2A-B show further detail of a filter module 60 according to one more particular embodiment as follows, and is shown after being locked and detached onto guidewire 40, and before (FIG. 2A) and after (FIG. 2B) being radially confined within a delivery lumen 56 of a delivery sheath 50.

More specifically, FIG. 2A shows filter member 61 in a radially expanded condition externally of sheath 50. A distally tapering circumferential wall 63 extends between an open proximal end 62, where it is supported by a ring or “loop”-shaped support member 64, and a distal end 66, where it is secured onto tubular support spine 70 that is locked onto wire 40 within inner lumen 72. In the radially expanded configuration shown in FIG. 2A distally extended from delivery sheath 50, the filter member 61 thus provides a pocket 65 that is open along proximal end 62, and closed at distal end 66. Wall 63 is substantially porous to such that normal physiologic blood components flowing into the pocket 65 will pass through wall 63, but whereas debris above a pre-determined dimension, such as from upstream (e.g. proximal relative to the module 60) interventions, will not pass and be captured within pocket 65.

FIG. 2B shows engagement of the module 60 within delivery lumen 56 of delivery sheath 50 subsequent to forming a filtering operation and with certain debris captured within filter member 61. As shown in one particular illustrative mode, such debris may provide increased profile to the collapsed condition of filter module 60, and thus it may be only partially engageable within the radially confining lumen 56 of sheath 50. However, in such circumstance, such may be removed as a system from the body, with the debris successfully filtered, captured, and removed.

FIG. 2B further shows more detail of the relationship between proximal support member 64 and its radially collapsed condition in the radially collapsed configuration for module 60 within delivery lumen 56 of sheath 50. Sheath 50 essentially grooms ring or “loop”-shaped support member 64 into a relatively linear orientation along longitudinal axis L, and radially collapses the otherwise open ring to a radially collapsed condition. This orientation allows for sufficient real estate within delivery lumen 56 to house support member 64 in the collapsed condition. Support member 64 may be provided in a slightly canted orientation in the radially expanded condition outside of sheath 50 in order to accommodate smooth relative advancement of sheath 50 over the ring-shape during the grooming process of radial engagement within lumen 56.

Support member 64 may be coupled to the annular end of the material sheet forming filter member 61 in a variety of modes apparent to one of ordinary skill, though the particular beneficial mode shown herein is described as follows for illustration (not shown). The annular end 62 includes a circumferential pouch formed by inverting or everting the end of the material sheet forming filter member 61 on itself and then bonding the inverted or everted edge to the wall, such as by heat bonding, material welding, solvent bonding, adhesive bonding, stitching, etc. the loop-shaped support member 64 may be positioned so as to be captured within the pouch as it is formed, or may be thereafter inserted therein, such as by leaving or forming un-bonded portions, e.g. apertures or ports into the pouch. This all may be accomplished for example by forming the member initially as a flat sheet and providing support member 64 as a partial looped region between two opposite free wire ends. Such arrangement leaves two opposite openings to the inverted or everted pouch along an axis at the edge of the sheet transverse to a long axis of the sheet. One of the top opposite free wire ends is inserted into the pouch and strung therethrough until the partial loop-shaped region is positioned within the pouch. By bringing the free opposite ends together, they may be bonded either together or to the support spine or tubing 70. In this arrangement, such free ends may be in a bent orientation transverse to the plane of the radius of curvature for the intermediate loop located within the pouch. In any case, the opposite longitudinal edges of the sheet are also brought together to form the partial tubular member, and may be either bonded together or bonded to spine 70 to form the filter module 60. In this arrangement, of course the sheet may be either post-processed, or cut along a pre-arranged correlate pattern, that allows for the shaped taper toward the distal end 66 which is rendered in a closed condition and secured to guidewire tracking and support spine 70.

The radially collapsed condition for support member 64 corresponds to a radially collapsed configuration for the overall filter assembly or module 60, which further includes a folded orientation for filter member 61. The radially expanded condition for support member 64 corresponds to a radially expanded configuration for filter assembly module 60, which includes an orientation for filter member 61 that spans across a substantial cross-section of the respective lumen within which it is deployed. In the particular beneficial embodiments shown, support member 64 is a material having substantial shape member, such as a metal alloy such as nickel-titanium alloy that demonstrates either shape member under thermal changes, or superelastic shape memory, during the change of conditions for the component. For example, the radially collapsed condition corresponds with a deformed condition of the material from a memory condition. The support member 64 is kept in the deformed condition within radially confining lumen 56 of sheath 50. Upon distal advancement therefrom, the force of radial confinement is removed, and thus support member 64 self-adjusts to the radially expanded or extended condition according to material recovery to the memory condition. Such memory condition and related memory shape may correspond with the shape shown for the radially expanded condition, or the memory shape may be something different and the support member 64 is still under some constraint or deformation therefrom even in the radially expanded condition. For example, the vessel wall itself may provide such restraint, and in fact such may allow for a range of lumens to be appropriately treated, as the support member 64 under external wall constraint may have varied radially expanded conditions with shapes on planes with different angles transverse to the longitudinal axis of the lumen in order to span the cross section of different diameters of lumens.

The particular shape and arrangement of filter member 61 in FIGS. 2A-B is illustrative and various other embodiments or variations are contemplated.

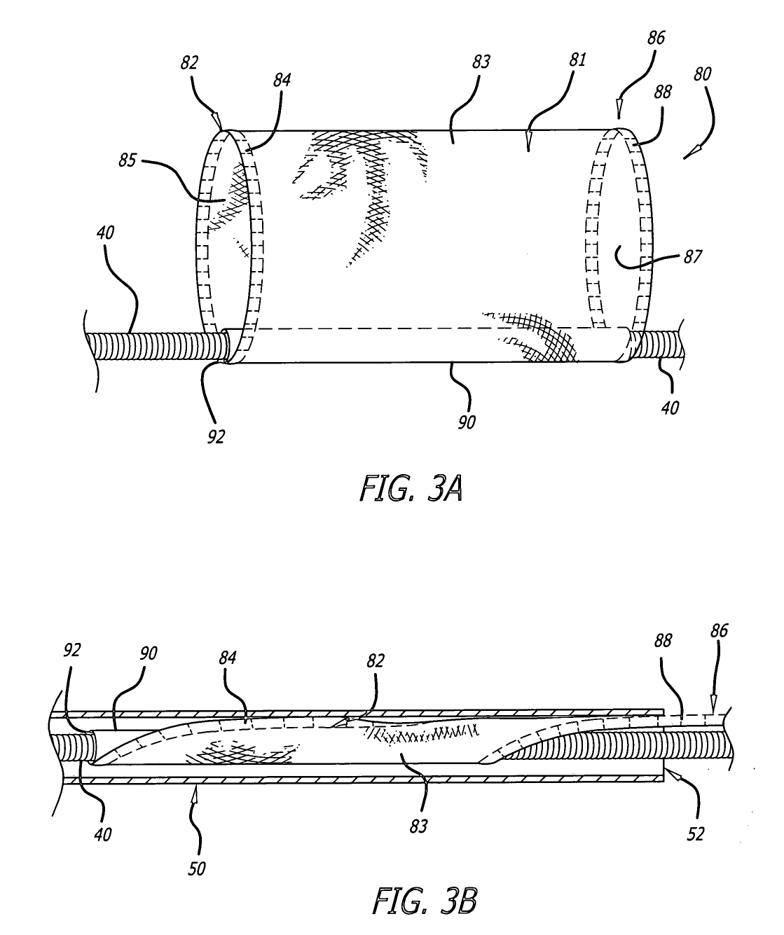

For example, FIGS. 3A-B show a particular arrangement that is modified from the embodiment of FIGS. 2A-B as follows. Filter module 80 is shown after being already locked and detached onto a guidewire 40, and includes a filter member 81 secured to a tubular support spine 90 that is locked onto guidewire 40 via inner lumen 92. Filter member 81 includes a circumferential filter wall 81 that extends between a proximal end 82 where it is coupled to a ring-shaped proximal support member 84, and a distal end 86 where it is coupled to a second ring-shaped distal support member 88. A further filter wall 87 is provided that spans across the circumferential confines of distal support member 88 at distal end 86. According to this arrangement, in the radially extended or expanded conditions for proximal and distal support members 84,88 correspond with the radially expanded configuration for filter member 81. In this condition, filter wall 83 is adapted to extend along a blood vessel wall, whereas filter wall 87 spans substantially across the vessel. Accordingly, pocket 85 is formed that ends distally at filter wall 87 as a “catch” or backstop against which debris of sufficient size is caught and prevented from passing.

These various support rings may be provided in a similar manner previously described above by reference to FIGS. 1-2B. Moreover, the relation and modes of operation between respective radially collapsed and expanded conditions may also be achieved and demonstrated in a similar manner, as shown by relative comparison between FIGS. 3A-B.

It is to be appreciated that, while the dual-support member embodiment just described is illustrative of many different configurations that may be provided, such particular embodiment also provides certain particular beneficial results. In one regard, doubling the radially expanding support rings doubles the opportunity for the filter assembly to properly engage the respective lumen's wall, and thus to catch all desired large debris flowing therethrough. Where only one such structure is provided to engage the wall, its sizing may not be optimal. However, as vessels taper, having two spaced filters may provide benefit in certain circumstances. Moreover, as they are shown of equal size in FIG. 3A, they may nevertheless be of different sizes or shapes, such as for example providing the distal support 88 with a smaller circumference than-proximal support 84, thus accommodating distally tapered lumens as described.

Various adjustable lock systems and methods are contemplated for providing the ability to lock an adjustable filter assembly at a selected location along a guidewire, and in particular for in-situ coupling.

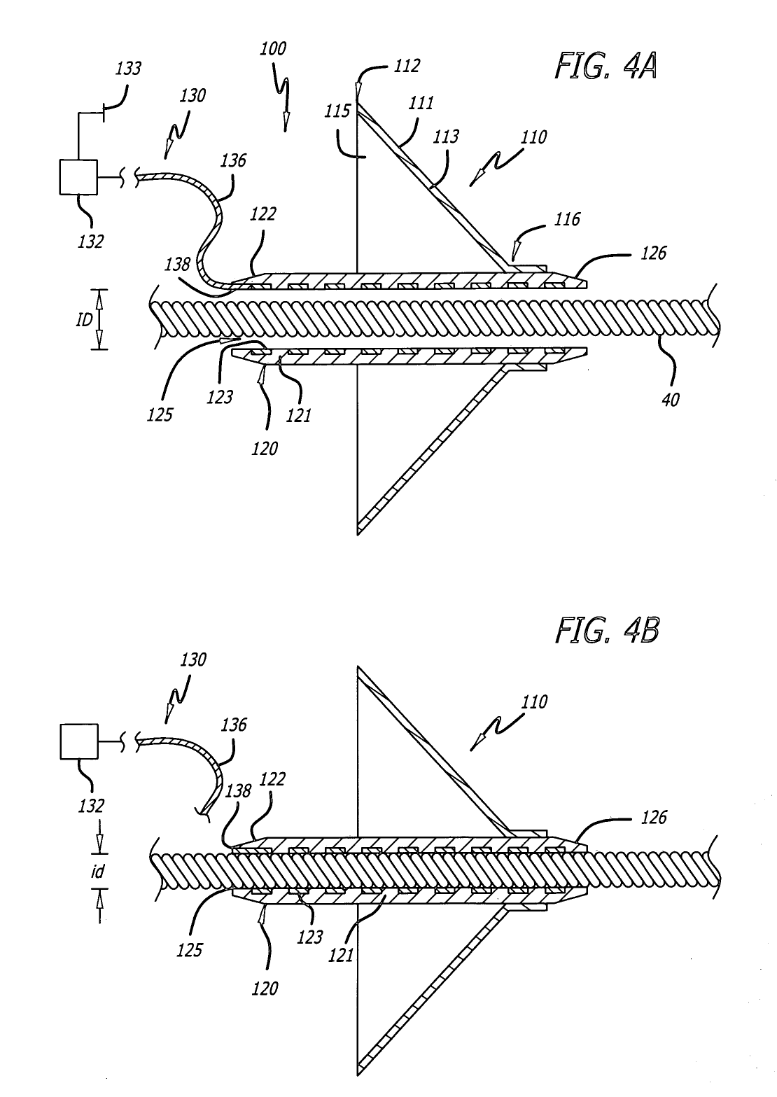

One particular example is illustrated in FIG. 4A as follows. System 100 is shown to include a filter assembly or module 110 with a filter member 111 engaged to a guidewire tracking member as a support spine 120 for filter member 111. Support spine 120 tracks over a guidewire via guidewire lumen 125 extending between opposite guidewire ports at proximal and distal ends 122, 126. Support spine 120 is constructed as a composite tubular member, with a coiled or braided filament support 123 imbedded or laminated within or onto a polymer or other material matrix 121. Filter member 111 is shown in a radially expanded configuration with a distally reducing tapered funnel-shaped wall 113 extending between a larger diameter open end 112 and a closed distal end 116 that is secured onto support spine 120 at distal end 126.

An adjustable lock assembly 130 includes an electrical source 132 coupled to the filament support wire 123 at a coupling joint 138 at proximal end 122. In the present embodiment wire 123 is constructed of a shape memory metal alloy that is exposed within lumen 125, and/or at one or both of ends 122,126, and is an electrical conductor. Electrical source 132 is also coupled to a second electrical lead 133 that is adapted to be coupled to the body. In this arrangement, a bipolar electrode system is created such that by actuating current source 132, current flows through a conductive path placed between wire filament 123 and electrode 133. Such may occur, for example, in an electrolytic bath, or in particular beneficial examples, using the patient as the conductor with the lead electrode 133 being for example a patch electrode positioned along the patient's back or other surface, and the wire filament 123 positioned with the module 110 along the guidewire 40 within the body at a desired filtering location. By allowing such current to flow, wire filament 123 is heated, and thus exhibits shape memory characteristics, which in this configuration is to recover to a memory shape having a smaller inner diameter id (FIG. 4B) than the inner diameter ID shown in the radially expanded condition in FIG. 4A.

The resulting locked condition for the adjustable lock assembly is shown in FIG. 4B, which shows member 120 with a reduced inner diameter id corresponding with the radially collapsed condition and that is shrunk with radial force onto the guidewire 40 to lock the components together. It is to be appreciated that, in order to achieve this reconfiguration in heat shrink mode, the remaining features of the composite forming tubular member 120, more specifically matrix 121 in which wire filament 123 is imbedded, must recover with the material recovery of the filament 123. Accordingly such matrix may be for example an elastomer which, for example, is itself in a deformed condition in the radially expanded configuration for member 120 shown in FIG. 4A. As such, the material recovery of filament 123 to the collapsed condition shown in FIG. 2B also corresponds to a material recovery for the matrix 121. In this particular arrangement, filament 123 in the radially expanded condition shown in FIG. 4A will generally have such radial strength in that condition so as to hold the mating matrix 121 in the elastically deformed state until the composite is adjusted to the radially collapsed configuration shown in FIG. 4B.

As also shown in FIG. 4B, the electrical conductor that coupled wire filament 123 and electrical source 132 is thereafter detached from member 120, as it is no longer required or desired in order to allow wire 40 and module 110 to now operate as a unitary assembly. Such detachment may be achieved for example using electrolytic detachment, such as by providing an electrolytic joint at joint 138. Electrolytic joints and related electrolytic detachment mechanisms may be deployed in modes similar to those previously described, such as for example for use in Guglielmi Detachable Coils (“GDC”) for delivering and detaching embolic coils for treating neuro-aneurysms. These prior disclosures may be suitably applied to this novel application, or may be suitably modified by one of ordinary skill based upon review of this disclosure and to the extent consistent with the objects provided hereunder.

Moreover, it is also to be appreciated that multiple conductor leads 136 may be used, which are actuated together to heat filament 123, but which are individually coupled to filament 123 with individual sacrificial electrolytic joints. Following the combined actuation of the leads and related heat shrink operation to lock member 120 onto wire 40, each individual lead may be thereafter energized with higher current that crosses the threshold at which the sacrificial joint electrolytically dissolves, thus detaching the array from the filament 123. This allows lower individual currents along each conductor lead to combine for an overall current sufficient to heat shrink the wound or braided filament conductor 123, which may be below the electrolytic threshold for any one sacrificial joint, and then higher current at each joint above that threshold for dissolving the respective joint.

Other adjustable lock mechanisms are contemplated.

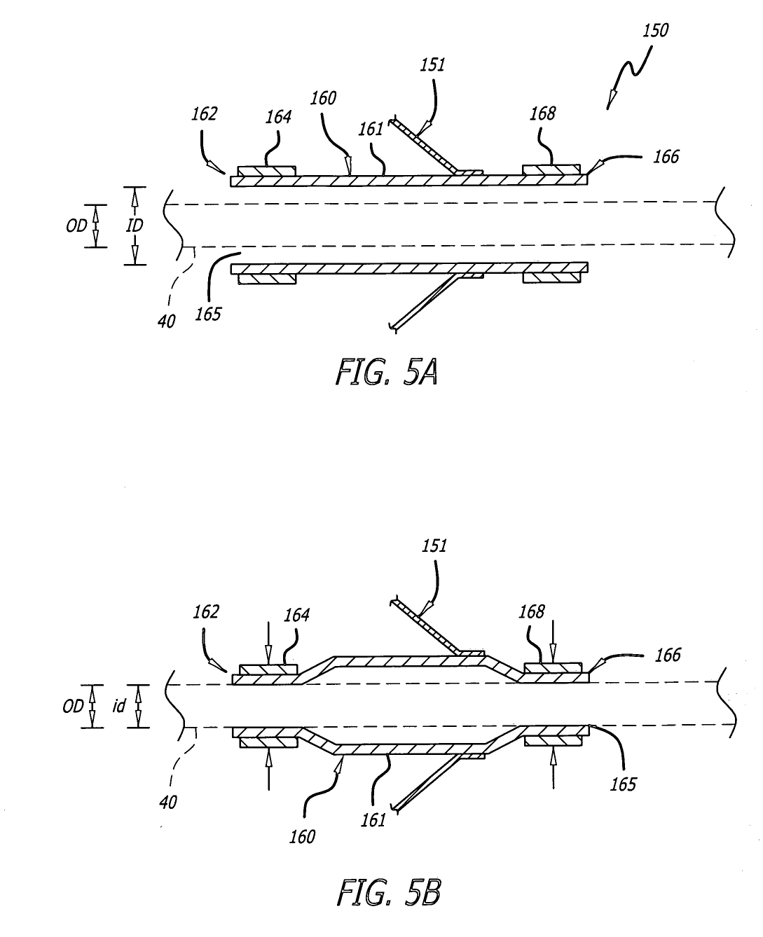

For example, FIGS. 5A-B show two respective modes of operation of a filter assembly 150 with a guidewire tracking support spine 160 with an adjustable lock mechanism shown adjusted between a radially expanded configuration with an inner diameter ID that is greater than the outer diameter OD of guidewire 40 as shown in FIG. 5A, and radially collapsed configuration with a reduced inner diameter id that is sufficient to engage outer diameter OD of guidewire 40, as shown in FIG. 5B.

More specifically, discrete adjustable diameter cuffs 164,168 are located on opposite ends 162,166 of the guidewire tracking support spine 160. Distal cuff 168 is located distally to the distal attachment point of filter member 151 onto support spine 160. In this arrangement, tubular wall 161 of support spine 160 is contracted by the distal cuff 168, but is not required to contract where filter member 151 is secured to the tubular wall 161.

Accordingly, tubular wall 161 is only required to provide the flexibility necessary for variable diameter sizing between the radially collapsed and expanded configurations at isolated regions. Where filter member 151 may be of such material construction not well adapted to be “shrunk” to a smaller diameter size, e.g. a preformed diameter that is non-elastomeric. Thus, it is further contemplated that tubular member 161 may have one construction along a mid-portion, and another more elastomerically adjustable construction at the locations where cuffs 164,168 are located, e.g. at the ends. In any event, these areas of tubular member 161 corresponding with cuffs 164,168 may be for example held elastomerically expanded by the coupling with expanded cuffs 164,168, for example via adhesive bonding or other mode of fixed engagement. Thus such regions may recover elastomerically to the radially collapsed configuration under the radial recovery force of cuffs 164,168 to the radially collapsed condition with an inner diameter id that matches outer diameter OD of guidewire 40.

Cuffs 164,168 may be constructed of shape memory material, such as nickel-titanium or other shape memory alloy, and may be adjustable upon change of temperature such as by applied external energy, or locally delivered energy such as via electrical current flow through the devices for resistance heating, or electrical coupling to the cuffs as electrically conductive monopolar electrodes for a completed circuit that includes the patient as described above, or light energy such as via optical fiber coupling or local light emitters cooperating with the assembly, or ultrasound crystals or other transducers located to heat the cuffs. Or, they may have transition temperatures below body temperature but above their storage temperature, e.g. room temperature (or they may be kept cold). Is such circumstance, by raising temperature above a critical transition temperature, they recover or “shrink” to the memory shape that is radially collapsed condition shown in FIG. 5B. Moreover, the cuffs may be elastomeric, e.g. superelastic, and held superelastically stretched in the open configuration by a mechanism such as an internally adjustable sheath that, upon removal from the inner diameter ID allows material recovery to the memory condition that shrinks the cuffs onto the guidewire. The cuffs may be solid, or have other shapes such as similar to various different types of stent designs previously disclosed for adjustable diameter use, e.g. with an interconnected pattern of struts and intervening voids, such as cut from a tube using a laser or etching, etc.

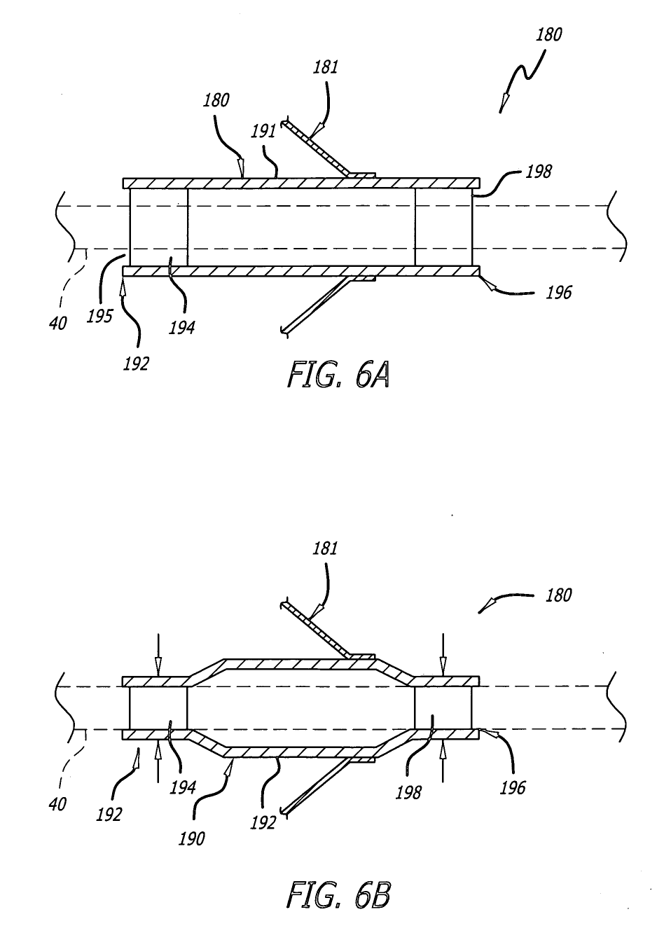

Other cuff-type embodiments are contemplated, as illustrated by way of example in FIGS. 6A-B. Here, a filter assembly module 180 is shown with a filter member 181 in partial cross-section view coupled to a tubular support spine 190 that has a tubular wall 191 providing a guidewire tracking member with a guidewire lumen 195 adapted in the radially expanded configuration shown to track over a guidewire 40. Proximal and distal cuffs 194,198 are located on each of two ends 192,196, respectively, of support spine 190 and within lumen 195 of tubular wall 191, versus on an exterior thereof as in the previous embodiment of FIGS. 5A-B. This configuration may be highly beneficial in the circumstance where tubular wall 191 at the ends coupled to cuffs 194,198 is to be held elastomerically radially expanded in the radially expanded condition for cuffs 194,198 as shown in FIG. 6A. By adjusting the cuffs to their respective radially collapsed condition shown in FIG. 6B, the outer tubular member 191 surrounding the cuffs is allowed to elastically recover with those cuffs.

It is to be appreciated that the various discrete cuff-type embodiments just described, though considered highly beneficial, are illustrative an other configurations, shapes, sizes locations, numbers, or respective arrangements of cuffs are contemplated. For example, as the cuffs are for selectively locking the overall filter assembly associated therewith onto the guidewire, a single cuff may suffice in certain arrangements. Such would simplify and reduce the cost of the overall arrangement, for example requiring only one actuator or coupler to deliver energy percutaneously to the cuff.

The provision of a selective lock assembly that provides for selective locking between a guidewire tracking filter assembly and the respectively tracked guidewire is to be considered broadly. Many different selective locks may be used. In fact, such may not be required to be integrated with the filter assembly itself, but may instead be a third assembly that cooperates with the guidewire and/or the filter assembly, or may be provided by the guidewire in a specialized design. However, by providing the lock other than by the guidewire, a wide variety of different guidewires may be used, providing substantial benefit to overall customization of procedures or to a treating physician's choices and techniques.

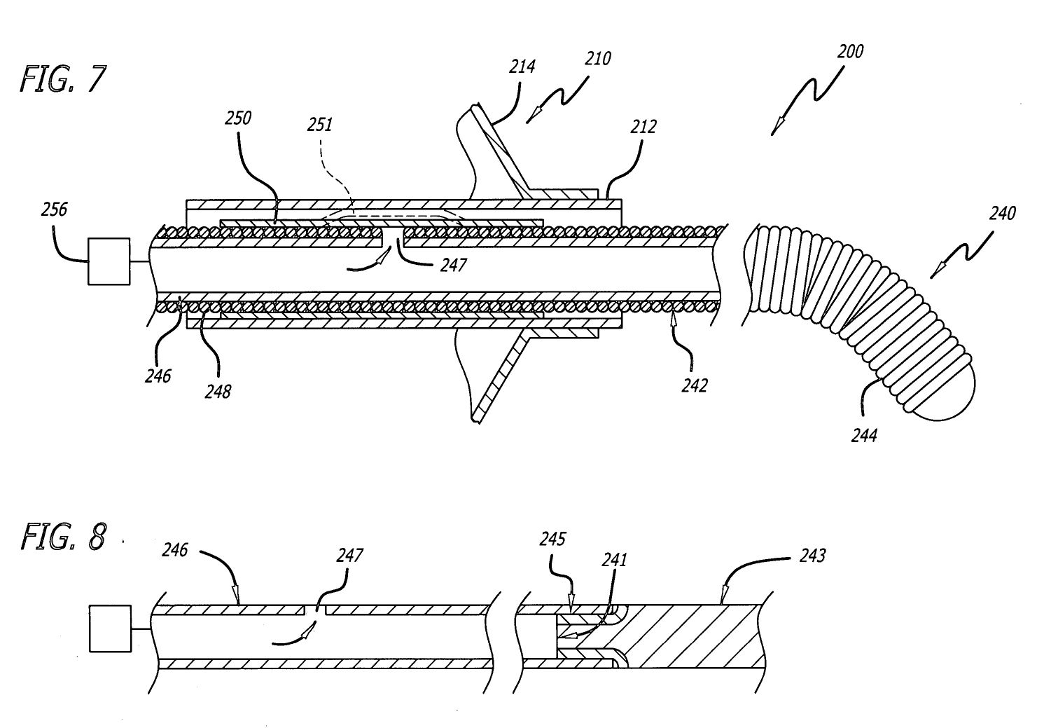

Nevertheless, the following embodiment provides further detail of a system 200 that provides an adjustable lock mechanism on a specialized guidewire 240 constructed according to the invention, as shown variously in FIGS. 7 and 8.

FIG. 7 shows system 200 to include a filter module 210 with a filter member 214 secured to a tubular support spine 212 that slideably engages guidewire 240 as a guidewire tracking member. Guidewire 240 has a shaped distal tip 244 extending from a body 242 constructed from an internal tubular member 246 within an external coil 248 and with a port or aperture 247 therethrough into an internal space confined by a pressure expandable outer cuff 250 that is secured to the outer coil 248 on either end of aperture 247. By coupling an external source of pressurizeable fluid 256 to the passageway within tubular member 246, outer cuff 250 expands under pressure via aperture 247 to radially expand, as shown at expanded cuff 251 in shadow, to sufficient dimension to radially engage the internal surface of tubular support spine 212 and thereby lock the guidewire 240 with filter assembly 210 in a friction fit under the normal force of the expanded cuff 250.

Various modes of construction may provide such a lock on a suitable guidewire to achieve the objective of locking into the interior of the guidewire tracking member of the filter assembly. Further detail of one construction of the core member suitable for use according to the embodiment shown in FIG. 7 is shown in FIG. 8. Tubular member 246 provides the proximal underlying guidewire chassis, and is constructed for example as a metal hypotube, e.g. stainless steel, nickel-titanium, etc., or from other suitable material to provide sufficient rigidity for torqueability and pushability requirements as generally understood for guidewires (e.g. may be high density polyethylene, or polyimide, or composite for example). Tubular member 246 is secured to a distal core member 243 by tapering the proximal end 245 of core member 243 to fit within the distal end hole or port 241 of the tubular member 246. They are thereafter secured in coaxial engagement, such as via welding, soldering, or use of adhesives. The tubular member 246 and core member 243 may be of like materials, or may dissimilar, such as by providing one of stainless steel, and the other of nickel titanium, in which case of course the chosen fixation means may require customization for these dissimilar metals, such as for example certain adhesives or solders (generally don't weld well-together, but such is a further contemplated mode and may be suitable in certain circumstances). Tip 244 typically includes a shapeable member internally therein, e.g. a flat ribbon or flat portion of the distal core 243, and the outer coil 248 in that region is typically radiopaque for x-ray visualization, e.g. platinum or tungsten or gold coil, with an atraumatic distal tip such as a smooth ball-shaped solder or weld cap.

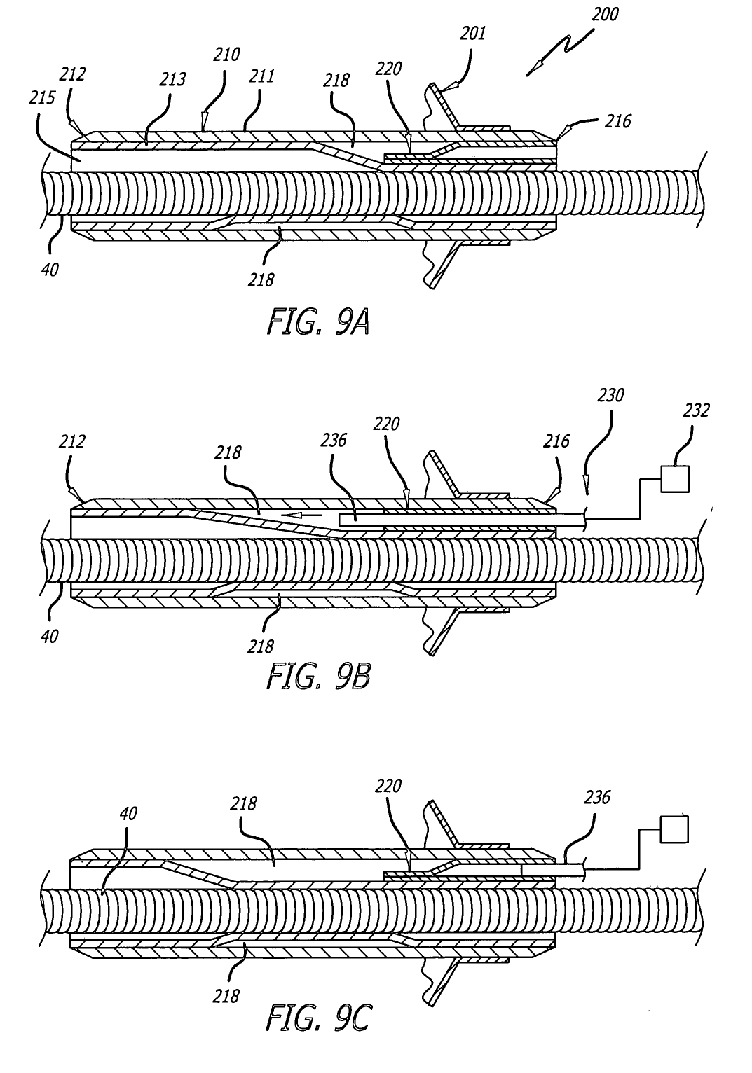

Another embodiment shown in FIGS. 9A-C provides a filter assembly 200 with a filter member 201 secured to a tubular spine member 210 that includes a tubular wall 211 that includes along its internal diameter an adjustable lock in another form of expandable membrane that is adjusted from an open position that is radially expanded relative to a guidewire 40 (FIG. 9A), to a closed position that is radially collapsed diameter relative to guidewire 40 (FIGS. 9B-C) as follows. Internal liner 213 is laminated or otherwise secured to tubular wall 211 in a manner providing a baffle or unlaminated pouch or reservoir 218 that is coupled to a duck-bill valve 220 along end 216 of the tubular member 210. An actuator assembly 230 includes a removably engaged inflation member 236 shown in FIG. 9B that is inserted within duck-bill valve 220 and is coupled to a source of pressurizeable fluid 232 externally of the patient. In this arrangement, pressurization of reservoir 218 expands the wall of the reservoir toward the interior of tubular wall 211, thus reducing the effective inner diameter to lock onto guidewire 40. Thereafter, inflation member 236 is removed at which time the duckbill valve 220 shuts from the eternal pressure experienced within reservoir 218, locking the assembly onto guidewire 40 permanently for unitary manipulation.

The particular arrangement shown is illustrative, and it is to be appreciated by one of ordinary skill that modifications may be made to suit a particular purpose without departing from the intended scope hereof. For example, duckbill valve 220 may be moved to the opposite side of tubular spine 210, to provide a different relative orientation for coupling to the actuator assembly 230 relative to the orientation of filter member 201. This may be modified in this manner for example for antegrade delivery such that the proximal end 212 is located downstream of the flow path into filter member 201, as would be apparent to one of ordinary skill.

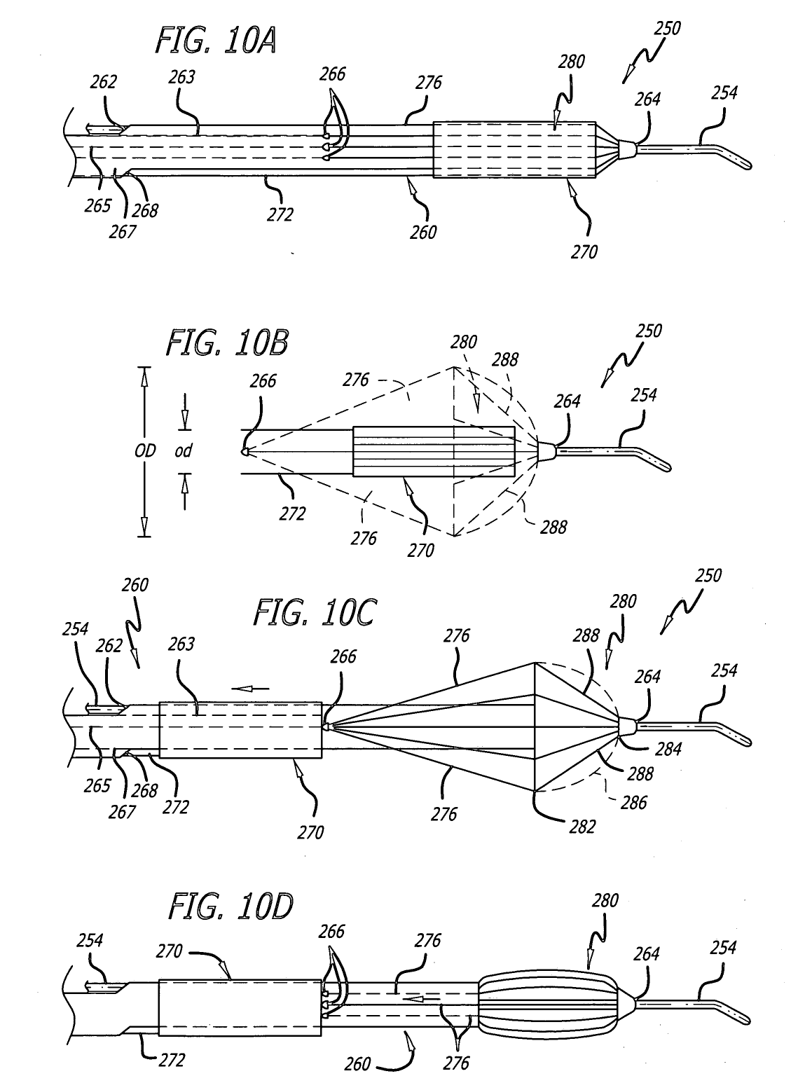

A further aspect of this disclosure is shown in FIGS. 10A-D and provides a filter system 250 that is a “rapid exchange” type of system that incorporates a moveable cuff 280 that is used to adjust the filter member 280 between radially collapsed and radially expanded configurations, respectively, and is further described by these and other features as follows.

Filter assembly 250 includes a filter member 288 that is secured to a support spine 260. Filter member 288 includes a sheet or membrane of similar shape to prior tapered embodiments with one open end and one closed end, but includes a plurality of circumferentially spaced splines 288 that have shape memory in a radially expanded condition that corresponds with a radially expanded configuration for the filter member 280, as shown in shadow in FIG. 10B. However, filter member 280 is held with splines 288 elastically deformed in a radially collapsed condition corresponding with a radially collapsed configuration for the filter member 280 when engaged within an interior of moveable cuff 270, as shown in FIGS. 10A and 10B.

As further shown in FIG. 10A, support member 260 includes a plurality of lumens within which are various coupling members having various functions and arrangements as follows. Lumen 268 carries a longitudinal spline 272 that is coupled at its distal end to adjustable cuff 270 and its proximal end (not shown) extends externally from the body for remote manipulation, either manually or by an actuator as would be apparent to one of ordinary skill. Another guidewire lumen extends between proximal and distal ports 262,264 that are both located on the distal end of the support member 260 on proximal and distal sides, respectively, of filter member 280. This provides for “rapid exchange” or “monorail” type of engagement over guidewire 254, such that the proximal end of guidewire 254 may be “backloaded” through distal port 264 and out proximal guidewire port 262 while the distal end of the guidewire 254 is located within the body, such as at or beyond the desired filter location.

A proximal portion of member 260 carries other lumens and extends proximally from proximal port 262 outside the body for remote manipulation to advance filter member 280 over guidewire 254 to the desired filter location. An array of distal ports 266 are circumferentially spaced around support member 260, and extend proximally therefrom to a proximal end portion of member 260 outside the body. Through distal ports 266 extend a plurality of tethers 276 that are coupled circumferentially around filter member 280, as shown in FIG. 10A and in shadow in FIG. 10B. Tethers 276 extend proximally from distal ports 266 along member 260, through multiple lumens, to a proximal location for remote manipulation externally of the body in order to control re-collapse of filter member 280 as explained in further detail below.

As illustrated in FIG. 10B, the filter member 280 is adjustable from a collapsed configuration with a collapsed outer diameter od to an expanded outer diameter OD. This is accomplished by proximal withdrawal of longitudinal spline 272 that withdraws cuff 270 proximally from filter member 280, releasing filter member 280 from radial confinement and allowing for memory recovery of support splines 288 to the expanded condition. This is shown in detail in FIG. 10C. In this expanded configuration, filter member 280 is adapted to span across a substantial cross-section of the body lumen where it is delivered, and is constructed with such porosity so as to filter components from blood flow above a pre-determined size, such as debris from upstream interventions. As such, filter member 280 may reconfigure its shape under the mass of such debris, as shown in shadow in FIG. 10C.

As further shown in FIG. 1C, tethers 276 are strung between the expanded proximal open end of filter member 280 in the expanded configuration and ports 266. Subsequent to the filtering operation in-vivo, the assembly 280 is to be withdrawn while capturing its contents therein. According to the present embodiment, this is accomplished by withdrawing tethers 276 through circumferential ports 266, which draws down the proximal open end of filter member 280 onto support member 260. As further shown in FIG. 10D, contents may leave a bulging drawn down condition for filter member 280, which may or may not fit into a respective delivery or introducer sheath; if it does fit, the system 250 is withdrawn from the body therethrough. If it does not so fit, then system 250 is removed together with the respective sheath.

It is to be appreciated that several advantages are achieved with the foregoing embodiments of FIGS. 10A-D in comparison with certain other embodiments. In one regard, the relatively short cuff 270 replaces the need for the adjustable delivery sheath 50 of prior embodiments that otherwise generally extends over all the deliver member components for the respective filter assembly and proximally through the vascular introduction site. This provides for a lower profile overall system. In another regard, the rapid exchange feature allows for substantial benefit for advancing the filter assembly distal to an intervention site, while freeing the guidewire to provide a rail proximal thereto for advancement of other catheters to the site of intervention, such as a balloon angioplasty, stenting, or atherectomy or thrombectomy device. In still a further regard, the retractable tethers provide a desirable mode for providing tight capture of the debris laden filter member onto the respective support member, aiding in low profiles for efficient withdrawal.

Accordingly, such features provided are considered independently beneficial broad aspects illustrated by the present embodiment of the invention, which are to be considered of independent value, in addition to their various combinations and sub-combinations.

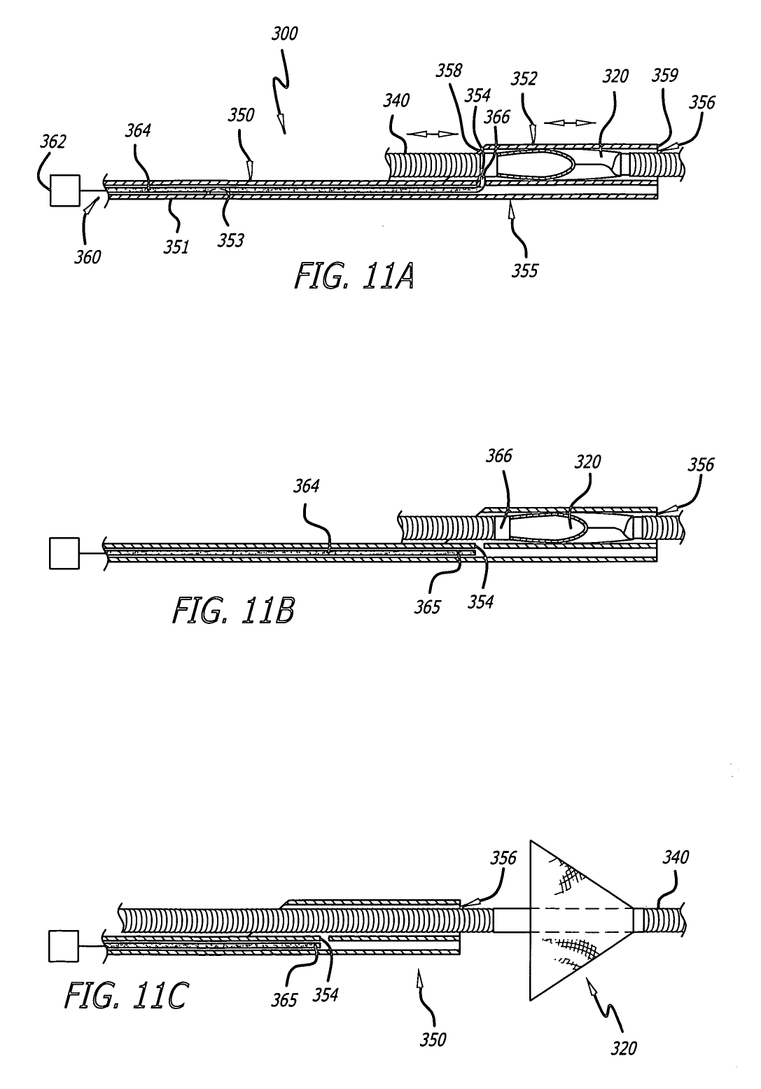

Rapid exchange features provided by the previous embodiments may also be incorporated in lockable filter assemblies similar to prior embodiments. One highly beneficial illustrative example is provided as follows by reference to FIGS. 11A-C.

FIG. 11A shows a filter system 300 that includes a filter assembly 320 cooperating with a delivery member 350 and guidewire 340. Delivery member 350 includes a proximal shaft portion 351 and a distal shaft portion 355. Proximal shaft portion 351 provides a tubular wall around a lumen 353 that further extends along distal shaft portion 355. However, distal shaft portion 355 further includes a second wall 352 that forms a second lumen 356 extending between proximal and distal ports 358,359, respectively that are both along the distal end portion of the member 350. Lumen 356 is thus a guidewire lumen that slideably engages a guidewire 340 in a “rapid exchange” or “monorail” fashion.

Filter assembly 320 is shown in FIGS. 11A-B in a radially collapsed configuration housed within lumen 356 and is engaged in a friction fit therein, and also held in place via coupler 364 located within lumen 353 and coupled to filter assembly 320 through port 354 between lumens 353,356. Filter assembly 320 may take many different forms, though in the particular embodiment shown is similar for illustration purposes to the embodiments shown and described by reference to FIGS. 1A-2B. Though not shown here in detail for clarity purposes, but by reference to those prior FIGS., filter assembly 320 includes a guidewire lumen through a support spine coupled to a filter member, and such guidewire lumen is also coaxially engaged over guidewire 340 as shown in FIGS. 11A-C.

Accordingly, by advancing proximal end portion of the delivery member 350 externally of the body, lumen 356 and the coaxially engaged guidewire lumen of the filter assembly 320 track over the guidewire to a desired location within the body. Thereafter, filter assembly 320 is adjusted to lock onto the guidewire 340, which may be for example via actuation of a lock mechanism with an applied energy via coupler 364. Thereafter, coupler 364 is detached from filter assembly 320 that has become unitary with guidewire 340 for manipulation purposes. This may be done for example by electrolytically detaching the coupling joint 366 (FIG. 11A) according in further examples to one or more of the various mechanisms herein described. Thereafter, distal end 365 of coupler 364 is retracted through port 354, as shown in FIG. 11B.

As shown in FIG. 11C, delivery member 350 may then be proximally retracted relative to guidewire 340 and locked filter member 320, thus removing filter member 320 from radial confinement and allowing filter member 320 to radially expand to the radially expanded configuration shown in FIG. 11C that is adapted to span across a lumen for filtering predetermined sized components of flow therethrough. Following completion of the filtering operation, the cuff portion forming lumen 356 may be again advanced distally to groom back down the filter assembly 320 to capture the contents therein for removal (not shown). Or, another mechanism may be employed to adjust the filter assembly 320 back to radially collapsed position and/or remove the filtered contents.

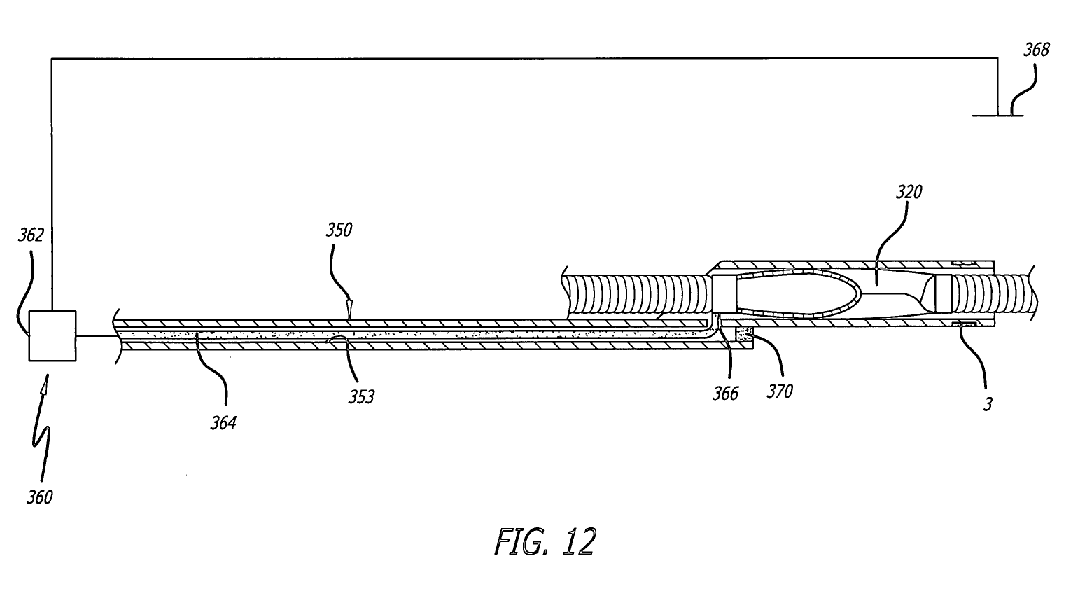

It is to be appreciated that the foregoing embodiments of FIGS. 11A-C are highly beneficial, though illustrative of broader contemplated aspects that may be achieve by many other modes without departing from the presently intended broad scope of the invention. For example, as shown in FIG. 12, lumen 353 may be terminated more closely distally adjacent to port 366. This results in a lower distal profile for the region of the overall assembly housing filter member 320, and thus one difference between FIGS. 11 and 12 is that the profile of the radially confining outer sheath area around the housed filter assembly is reduced as the lumen that carries the conductor lead to the filter device locking cuff is terminated immediately distally from the coupling. This provides a substantial benefit for example for distal embolic filters that must first cross occlusions prior to being deployed for filtering.

As also shown for further illustration in FIG. 12, one or more markers 370 may be provided on delivery member 350 for the purpose of providing indicia regarding the relative position of the delivery member 350 with respect to the underlying filter member 320. It is also to be appreciated that the particular configuration shown for filter member 320 is illustrative for clarity in the present embodiments, and other forms of filter member may be employed, such as for example various of the other embodiments herein described as would be appropriately applied here to one of ordinary skill in the art. Or, other filter assemblies otherwise known, anticipated or suggested, or otherwise obvious to one of ordinary skill may be suitably modified or applied to the present embodiments.

The “filter” assemblies herein referred to and by reference to the figures may incorporate various features and modes of operation and use of other previously disclosed filter assemblies, though modified according to various of the novel aspects of the present invention herein described by illustration through the embodiments below. Examples of such acceptable filter materials and designs, in addition to various modes of use and in combination with other devices in overall medical treatment systems, are provided in the various documents previously herein incorporated in their entirety by reference thereto in the “Background” section above.

Generally, various mechanical, electromechanical, or opto-mechanical modes may be used to adjust an embolic filtering module to alternatively slide or lock onto a guidewire. Several of the FIGS. provided herein schematically show an external energy source, such as a current source, coupled via a conductor to a portion of the filter device that acts as an electrode. The electrode, in the monopolar embodiments shown, coupled via the patient's tissues to a patch electrode to complete a circuit. Alternating RF frequency of sufficient amplitude will heat the electrode at the filter device to cause rise in temperature for shrinking down of a shape memory member onto the guidewire. The shape memory member may be the same member that serves as the electrode, such as a cuff, coil, or braid coupled to a support tube on which the filter assembly is secured.

The conductor of the various embodiments, e.g. as for example coupling member 36 shown in FIGS. 1A-C, may be detachable, as such component becomes unnecessary after locking the respective filter module onto the wire. This may be done using a sacrificial electrolytic link between the wire and the adjustable member, in a similar arrangement for example as previously disclosed with respect to commercially available detachable embolic coils, such as to occlude AVM's, fistula's, or aneurysms.

Monopolar embodiments are variously provided for illustration of the electrically energized embodiments. However, where not shown they are to be considered applicable. Moreover, other embodiments are contemplated. Bi-polar arrangements or “closed loop” electrical circuitry (e.g. resistance heating) can be used to electrically heat the material to cause the adjustment that locks the filter device onto the guidewire. Other heating modes include ultrasound, light, thermal conduction, or other energy sources either integrated into the filter device itself, or coupled thereto. For example, an ultrasound crystal coupled to the inner diameter of an outer radially confining sheath may be used to sufficiently heat the adjustable member for locking (not shown). Or, where electrical lead coupling is shown, a light fiber may be replaced to couple light energy such as laser or UV to the adjustable member to shrink it down or otherwise reshape it to cause the desired locking.

It is also to be appreciated that adjustable lock mechanisms utilizing heat shrink materials and modes may vary as to certain particular features. For example, FIGS. 4A-B show an adjustability along the entire length of a support tubular member for locking onto the guidewire, whereas FIGS. 5A-6B show alternative embodiments with more localized regions of radial adjustability for guidewire locking. Moreover, other modes than those shown among the embodiments may be used for locking, including for example radial or longitudinal mechanical forces to adjust shapes of various members, such as for example twisting or longitudinally tensioning a coil or braid to adjust the inner or outer diameter. In further modes not shown, locking may be achieved with local delivery of a small amount of adhesive, such as two-part component mixed in situ or within appropriate time of delivery before such “sets” for bonding. Or, a portion of plastic may be melted onto the guidewire to provide coupling with the respective filter member (or visa versa).

In another regard, various of the embodiments, such as for example among FIGS. 1A-6B, show an outer adjusting sheath as separate from the energy coupling system that provides for the locking mechanism. However, they may be considered separate parts of a cooperative control system that provides multiple functions to operate the filter device to provide medical care in combination with a guidewire and other inter-cooperating components. Such control system may include a more integrated assembly of component parts, such as shown in FIGS. 10A-12.

Embolic filter devices according to the invention, and by reference to the various illustrative embodiments, may be constructed from various materials and to various dimensions as would be apparent to one of ordinary skill based at least in part upon review of this disclosure. However, for illustration, it is contemplated in particular embodiments that the filter devices may be adapted to operate over guidewires having outer diameters of 0.010″, 0.014″, 0.018″, and 0.035″. Moreover, kits of such devices may be provided, each being particularly adapted for use over a different sized guidewire, or each having the filter assembly being particularly adapted for use to filter blood flowing within arteries of varied dimensions.

Various references are herein made to “interventions” or interventional devices for use with the filter system(s) of the present invention. While many substitutes may be made according to one of ordinary skill, examples include angioplasty, stenting, and atherectomy devices and methods for recanalization of occlusions.

For purpose of further illustration, one mode of using certain of the embolic filtering system embodiments of the invention is described as follows for a more complete understanding by reference to a recanalization procedure in a carotid artery occlusion.

Initially, a guidewire is placed using conventional techniques across the carotid artery occlusion, typically using a femoral or radial artery access technique with antegrade delivery to the occlusion site (often including use of a guiding catheter, and often an introducer sheath). A Seldinger technique may be used for example to provide such luminal access. Next, an embolic filter system is engaged over the guidewire by “back-loading” the guidewire through a guidewire lumen provided through a tubular support member of the embolic filter device. This is done with a radially confining sheath positioned over the embolic filter assembly to keep it in a radially collapsed and folded condition. The system is slideably advanced over the guidewire and across the occlusion site until the embolic filter device is located at a desired distal position for filtering. Then it is activated to lock it onto the wire in-situ at the distal position. Next, the assembly with radially confining sheath is withdrawn proximally to release the filter assembly from confinement, allowing shape memory of the assembly to expand it to an expanded configuration sufficient to span across a majority of the artery at the distal position for filtering. Where a coupling is provided directly to the embolic filter device for locking. e.g. via an electrical coupling lead, the coupler or lead is detached prior to proximal withdrawal. The various components of the control system may be withdrawn completely off from the guidewire, after which interventional device is replaced thereon and advanced to the occlusion for recanalization.

During the intervention, the filter is located and expanded to filter emboli released into downstream flow—this may also be left in place for sufficient time after intervention to catch further emboli.

In any event, when appropriate according to a treating physician, the filter assembly is adjusted back to a radially collapsed condition to capture the emboli filtered from the downstream blood flow. This may be done by again advancing a radially confining sheath over the wire and over the filter, such as by using the first control system a second time, or with a second outer sheath. Or, a pull wire or multiplicity thereof may be used to pull down support member(s) supporting the filter assembly in the expanded configuration. Depending upon the amount of emboli captured, all of the collapsed filter assembly may not be small enough to fit into an outer sheath, which case the entire system may need to be withdrawn over the guidewire and from the body. Otherwise, the collapsed filter may be withdrawn through the outer sheath, or filter and outer sheath together withdrawn within a guiding catheter guide lumen.

The various embodiments described above are generally intended for use in overall embolic filtering systems intended to be used in cooperation with other devices to filter primarily emboli from blood flowing through vessels downstream from an intervention site. Certain reference is made to specific beneficial applications for the purpose of illustration, but such specified applications are not intended to be limiting. For example, reference to the embolic filters of the invention is often specified for use in distal filtering downstream from interventions as the most frequent type of filtering used in conventional interventions. However, other filters for all uses may be made according to the various embodiments herein described, including for example proximal filters. In addition, it is also contemplated that other regions of the body may be effectively filtered than those specifically described herein, such as other body lumens including for example veins, gastrointestinal lumens, urinary lumen, lymph ducts, hepatic ducts, pancreatic ducts, etc. In addition, whereas many different filters may be used, the coupling of filters to guidewire tracking or locking chassis per the embodiments may be done by any conventional acceptable substitute modes. In addition, various locking mechanisms have been described for purpose of providing a detailed illustration of acceptable modes of making and using the invention. However, other locking modes may be employed without departing from the scope of the invention.

Where “proximal” or “distal” relative arrangements of components, or modes of use, are illustrated, other arrangements are contemplated though they may not be shown. For example, where various of the embodiments are adapted for antegrade use, they may be modified for retrograde delivery and use. In addition, proximal filtering may be accomplished according to the invention, such as by positioning a filter device proximal to an occlusion and using applied retrograde flow to wash emboli proximally into the filter.

Various modifications may be made to the previously disclosed embodiments above without departing from the scope of the present invention which is intended to be read as broad as possible with regard to the intended objectives described herein and without impinging upon what is already known in the art. Many examples of such modifications have been provided as illustrative and are not intended to be limiting, though significant value may be had in relation to certain such specific modifications or embodiments. Where particular structures, devices, systems, and methods are described as highly beneficial for the primary objective herein to provide adjustable embolic filters, other applications are contemplated both in medicine and otherwise in and out of the body. For example, various of the adjustable locking assemblies described may be found highly beneficial for use in locking other devices and assemblies over guidewires or other internal structures. In another example, the embodiment showing a guidewire with expandable member used to lock a filter thereon may be used to internally lock onto other outer coaxial structures.

Other applications may include adjustable annular collars used to lock down over centrally located devices extending within their bore. Another additional use for further illustration includes use of such adjustable locking members and related assemblies to graft two adjacent work pieces together, such as in a medical application to attach two pieces of bone together as a bone grafting tool.

The various detailed descriptions of the specific embodiments may be further combined in many differing iterations, and other improvements or modifications may be made that are either equivalent to the structures and methods described or are obvious to one of ordinary skill in the art, without departing from the scope of the invention. The illustrative examples therefore are not intended to be limiting to the scope of the claims below, or with respect to the Summary of the Invention, unless such limitation is specifically indicated.

1-3. (canceled)

4. An embolic filter system, comprising:
a delivery member with an elongate body with a proximal end portion and a distal end portion;
a filter assembly with a filter member having a wall with a substantially annular passageway around a circumference, and with a loop-shaped member coupled to the filter member within the annular passageway and along the circumference;
wherein the loop-shaped support member comprises a superelastic material and is adjustable between a radially collapsed condition corresponding with an elastically deformed condition for the superelastic material and a radially expanded condition according to material recovery from the elastically deformed condition to a superelastic shape memory condition for the superelastic material;
wherein adjusting the support member between the radially collapsed condition to the radially expanded condition at least in part adjusts the filter member between a radially collapsed configuration and a radially expanded configuration, respectively;
wherein the distal end portion of the delivery member is adapted to deliver the filter assembly to a location within a body lumen in a body of a patient with the support member radially confined in the radially collapsed condition and the filter member in the radially collapsed configuration and by manipulating the proximal end portion of the delivery member externally of the patient;
wherein the support member and filter member are adjustable from the radially collapsed condition and radially collapsed configuration, respectively, to the radially expanded configuration and radially expanded configuration, also respectively, at the location;
wherein the filter member in the radially expanded configuration at the location spans across a substantial cross-section of the body lumen and is adapted to filter components of fluid flowing through the body lumen at the location above a predetermined size.

5. An embolic filter system, comprising:
a delivery member with an elongate body having a proximal end portion and a distal end portion with a longitudinal axis, and a lumen extending between proximal and distal ports each being located along the distal end portion;
a filter assembly with a filter member that is adjustable from a radially collapsed configuration to a radially expanded configuration according to a material memory recovery from an elastically deformed condition toward a memory condition;
wherein the filter assembly in the radially collapsed configuration is radially confined within the lumen and is adapted to be delivered therein to a location within a body lumen in a body of a patient;
wherein the radially confining lumen has an adjustable position relative to the filter assembly at the location so as to release the filter assembly therefrom such that the filter member self-expands from the radially collapsed configuration to the radially expanded configuration at the location;
wherein the filter member in the radially expanded configuration at the location spans across a substantial cross-section of the body lumen; and
wherein the filter member in the radially expanded configuration at the location is adapted to filter components of fluid flowing through the body lumen at the location above a predetermined size.

6. A method for filtering emboli from fluid flowing across a location within a body lumen in a patient, comprising:
delivering a filter assembly in a radially collapsed configuration over a guidewire to the location;
locking the filter assembly onto the guidewire at the location;
adjusting the filter assembly locked onto the guidewire from the radially collapsed configuration to a radially expanded configuration at the location; and
wherein the filter assembly in the radially expanded configuration at the location spans across a substantial cross-section of the body lumen and is adapted to filter the emboli from the fluid flowing across the location.

7. A method for filtering emboli from fluid flowing across a location within a body lumen in a patient, comprising:
delivering a filter assembly with a delivery member in a radially collapsed configuration over a guidewire to the location;
detaching the filter assembly from the delivery member at the location;
adjusting the filter assembly from the radially collapsed configuration to a radially expanded configuration at the location; and
wherein the filter assembly in the radially expanded configuration at the location spans across a substantial cross-section of the body lumen and is adapted to filter the emboli from the fluid flowing across the location.

8. A method for filtering emboli from fluid flowing across a location within a body lumen in a patient, comprising:
positioning a filter assembly in a radially collapsed configuration within a capture lumen of a radially confining cuff having an adjustable position relative to the filter assembly;
providing the filter assembly in the radially collapsed configuration within the adjustable radially confining cuff along a distal end portion of a delivery member;
delivering the distal end portion of the delivery member and filter assembly in the radially collapsed condition within the cuff to the location;
adjusting the filter assembly from the radially collapsed configuration to a radially expanded configuration at the location by adjusting the relative position of the cuff relative to the filter assembly such that the filter assembly is released from radial confinement and self-expands according to material memory to the radially expanded condition;
wherein the filter assembly in the radially expanded configuration at the location spans across a substantial cross-section of the body lumen and is adapted to filter the emboli from the fluid flowing across the location; and
wherein the capture lumen extends along a length between proximal and distal ports and is adapted to be located entirely within the body lumen.

9-13. (canceled)

14. The system of claim 70, wherein:
the locking member comprises an annular wall with a circumference around an inner passageway with an inner diameter;
the first shape comprises a first inner diameter; and
the second shape comprises a second inner diameter that is less than the first inner diameter.

15. The system of claim 14, wherein the locking member comprises a composite tube-shaped member.

16. The system of claim 15, wherein the composite tube-shaped member comprises a nickel-titanium support structure coupled to an elastomeric wall.

17. The system of claim 14, wherein the locking member comprises an annular cuff coupled to a portion of a tubular member.

18. The system of claim 14, wherein the adjustable lock assembly comprises a plurality of said locking members, each locking member comprises a discretely located annular cuff coupled to a portion of a tubular member.

19. The system of claim 70, wherein:
the locking member comprises a shape memory material that is adjustable between the first shape and the second shape according to material recovery to a memory shape upon a change of temperature above a predetermined temperature.

20. The system of claim 70, wherein:
the adjustable lock assembly is adapted to apply an electrical current to the locking member at the location; and
the locking member is adapted to heat above the predetermined temperature in response to the applied electrical current at the location.

21. The system of claim 70, wherein
the adjustable lock assembly comprises an electrical conductor and is adapted to form a monopolar electrical lead at the location within the lumen of the patient;
the adjustable lock assembly is adapted to apply a radiofrequency (RF) current between the electrical conductor and a second electrical lead coupled to the patient; and
the locking member is adapted to heat above a predetermined temperature in response to the applied RF current at the location.

22. (canceled)

23. The system of claim 70, wherein
the locking member comprises an electrical conductor and is adapted to be electrically coupled to an electrical current source in a closed loop electrical circuit through the locking member;
wherein the locking member is adapted to undergo resistance heating above the predetermined temperature in response to an applied electrical current in the closed loop electrical circuit.

24. (canceled)

25. The system of claim 70, wherein the adjustable lock assembly further comprises:
a convection heater thermally coupled to the locking member;
wherein the convection heater is adapted to be actuated to convect sufficient heat toward the locking member so as to raise the temperature of the locking member above a predetermined temperature at the location.

26. The system of claim 70, wherein the adjustable lock assembly further comprises:
an ultrasound emitter that is adapted to be ultrasonically coupled to the locking member sufficient to raise the temperature of the locking member above the a predetermined temperature at the location.

27-28. (canceled)

29. The system of claim 70, wherein the adjustable lock assembly further comprises a microwave element that is adapted to inductively heat the locking member at the location.

30-31. (canceled)

32. The system of claim 70, further comprising:
an optical coupler that is adapted to couple light from a light emitter and to transmit the light to the adjustable lock assembly.

33. (canceled)

34. The system of claim 70, wherein:
the locking member comprises a superelastic material;
the first shape corresponds with a superelastically deformed condition for the superelastic material; and
the adjustment from the first shape to the second shape corresponds with a superelastic material recovery from the deformed condition to a superelastic memory shape condition.

35-37. (canceled)

38. The system of claim 70, wherein:
the locking member comprises an inflatable bladder that is adapted to be located between the filter assembly and the distal end portion of the guidewire at the location and to couple to a source of pressurizeable fluid externally of the patient; and
the inflatable bladder is adjustable from a collapsed condition that is substantially uninflated and corresponds with the open configuration for the adjustable lock assembly to an inflated condition that is inflated with fluid from the source and corresponds with the locked configuration for the adjustable lock assembly.

39-56. (canceled)

57. The system of claim 69, wherein:
the delivery member comprises a proximal end portion and a distal end portion that is detachably coupled to the filter assembly; and
the proximal end portion of the delivery member comprises a wire.

58. The system of claim 69, wherein:
the delivery member comprises a proximal end portion and a distal end portion that is detachably coupled to the filter assembly; and
the proximal end portion of the delivery member comprises a tubular member.

59-61. (canceled)

62. The system of claim 69, wherein the filter member comprises a porous polymer material.

63. The system of claim 62, wherein the porous polymer comprises a porous polytetrafluoroethylene material.

64. The system of claim 69, wherein the filter member comprises an expandable cage of filament members.

65-66. (canceled)

67. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration; and
wherein the adjustable lock assembly is adjustable at the location from the open configuration to the locked configuration that is adapted to lock the filter assembly at a selected position onto a distal end portion of the guidewire at the location.

68. An embolic filter system, comprising:
a delivery member;
a filter assembly with a filter member;
wherein the delivery member is detachably coupled to the filter assembly;
wherein the filter assembly is adapted to be advanced over a guidewire with the delivery member to a location within a body lumen in a body of a patient; and
wherein the delivery member is adapted to be detached from the filter assembly at the location.

69. An embolic filter system, comprising:
a delivery member;
a filter assembly with a filter member and an adjustable lock assembly;
wherein the adjustable lock assembly is adjustable between an open configuration and a locked configuration;
wherein the filter assembly is adapted to be advanced over a guidewire with the delivery member to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the filter assembly is adapted to be detached from the delivery member at the location.

70. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration; and
wherein the adjustable lock assembly is adjustable at the location from the open configuration to the locked configuration that is adapted to lock the filter assembly at a selected position onto a distal end portion of the guidewire at the location; and
wherein the adjustable lock assembly comprises a locking member that is adjustable between a first shape and a second shape, the first shape corresponding with the open configuration, and the second shape corresponding with the locked configuration.

71. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the adjustable lock assembly is adapted to deliver a material between the filter assembly and the guidewire that secures the filter assembly to the guidewire in the locked configuration.

72. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the adjustable lock assembly comprises a material and is adapted to melt the material between a first condition corresponding with the open configuration and a second condition corresponding with the locked configuration and that is adapted to secure the filter assembly to the guidewire.

73. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the adjustable lock assembly in the locked configuration comprises an adhesive located between the filter assembly and the guidewire and securing the filter assembly to the guidewire.

74. An embolic filter system, comprising:
a delivery assembly with a delivery member;
a filter assembly with a filter member and an adjustable lock assembly;
wherein the adjustable lock assembly is adjustable between an open configuration and a locked configuration;
wherein the filter assembly is adapted to be advanced over a guidewire with the delivery member to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location;
wherein the filter assembly is adapted to be detached from the delivery member at the location;
wherein the delivery assembly further comprises a capture lumen extending between proximal and distal ports located along the distal end portion of the delivery assembly, respectively;
wherein the filter assembly in the radially collapsed configuration is radially confined within the capture lumen and is adapted to be delivered therein to the location; and
wherein the capture lumen has an adjustable position relative to the filter assembly at the location so as to release the filter assembly therefrom such that the filter member self-expands from the radially collapsed configuration to the radially expanded configuration at the location.

75. An embolic filter system, comprising:
a delivery member;
a filter assembly with a filter member;
wherein the delivery member is detachably coupled to the filter assembly;
wherein the filter assembly is adapted to be advanced over a guidewire with the delivery member to a location within a body lumen in a body of a patient;
wherein the delivery member is adapted to be detached from the filter assembly at the location;
wherein the delivery member is detachably coupled to the filter assembly via an electrolytically sacrificial joint; and
wherein the electrolytically sacrificial joint is adapted to be coupled to a current source in an electrical circuit that includes the joint at the location such that the delivery member is detachable from the filter assembly by electrolytic dissolving of the joint.

76. An embolic filter system, comprising:
a delivery member;
a filter assembly with a filter member;
wherein the delivery member is detachably coupled to the filter assembly;
wherein the filter assembly is adapted to be advanced over a guidewire with the delivery member to a location within a body lumen in a body of a patient;
wherein the delivery member is adapted to be detached from the filter assembly at the location;
wherein the delivery member comprises multiple conductors, each conductor being detachably coupled to the filter assembly via an electrolytically sacrificial joint; and
wherein each electrolytically sacrificial joint is adapted to be coupled to a current source in an electrical circuit that includes the joint at the location such that the distal end portion is detachable from the filter assembly by electrolytic dissolving of each joint.

77. An embolic filter system, comprising:
a delivery member;
a filter assembly with a filter member;
wherein the delivery member is detachably coupled to the filter assembly;
wherein the filter assembly is adapted to be advanced over a guidewire with the delivery member to a location within a body lumen in a body of a patient;
wherein the delivery member is adapted to be detached from the filter assembly at the location; and
the delivery member is adapted to be mechanically detached from the filter assembly at the location.

78. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the adjustable lock assembly is adapted to mechanically engage the guidewire with an applied force onto the guidewire in the locked configuration.

79. An embolic filter system, comprising:
a filter assembly with a filter member and an adjustable lock assembly;
wherein the adjustable lock assembly is adjustable between an open configuration and a locked configuration;
wherein the filter assembly is adapted to slideably engage and track over a guidewire to a location within a body lumen in a body of a patient with the adjustable lock assembly in the open configuration;
wherein the adjustable lock assembly is adjustable at the location from the open configuration to the locked configuration that is adapted to lock the filter assembly at a selected position onto a distal end portion of the guidewire at the location; and
wherein the adjustable lock assembly comprises a pressurizeable bladder that is adapted to mechanically engage the guidewire with an applied force onto the guidewire in the locked configuration.
